SEQUENCE LISTING (4 sequences)

SEQ ID NO: 1 - DNA, 3822 bp, synthetic construct
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 36556 bp, synthetic construct
  source 1..36556: mol_type = other DNA
atcatcaaataatatacctcaaacttttggtgcgcgttaatatgcaaatgagctgtttgaatttggggatgcggggctgtgattggctgtgggagcggcgaccgttaggggcggggcgggtgacgttttgatgacgtgtttgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgtaggcgtcagctgatcgccagggtatttaaacctgcgctctctagtcaagaggccactcttgagtgccagcgagtagagttttctcatccgcgccgcgagtcagatctacactttgaaagatgaggcacctgagagacctgcccggtaatgttttcctggctactgggaacgagattctggaactggtggtggacgccatgatgggtgacgaccctcctgagccccataccccatttgaggcgccttcgctgtacgatttgtatgatctggaggtggatgtgcccgagaacgaccccaacgaggaggcggtgaatgatttgtttagcgatgccgcgctgctggctgccgagcaggctaatacggactctggctcagacagcgattcctctctccataccccgagacccggcagaggtgagaaaaagatccccgagcttaaaggggaagagcttgacctgcgctgctatgaggaatgcttgcctccgagcgatgatgaggaggacgaggaggcgattcgagctgcagcgaaccagggagtgaaagcagcgagcgagggctttagcctggactgtcctactctgcccggacacggctgtaagtcttgtgaatttcatcgcatgaatactggagataagaatgtgatgtgtgccctgtgctatatgagagcttacaaccattgtgtttacagtaagtgtgattaactttagttgggaaggcagagggtgactgggtgctgactggtttatttatgtatatgttttttatgtgtaggtcccgtctctgacgcagatgagacccccacttcagagtgcatttcatcacccccagaaattggcgaggaaccgcccgaagatattattcatagaccagttgcagtgagagtcaccgggcggagagcagctgtggagagtttggatgacttgctacagggtggggatgaacctttggacttgtgtacccggaaacgccccaggcactaagtgccacacatgtgtgtttacttaaggtgatgtcagtatttatagggtgtggagtgcaataaaatccgtgttgactttaagtgcgtgttttatgactcaggggtgggactgtgggtatataagcaggtgcagacctgtgtggtcagttcagagcaggactcatggagatctggactgtcttggaagactttcaccagactagacagctgctagagaactcatcggagggagtctcttacctgtggagattctgcttcggtgggcctctagctaagctagtctatagggccaaacaggattataaggatcaatttgaggatattttgagagagtgtcctggtatttttgactctctcaacttgggccatcagtctcactttaaccagagtattctgagagcccttgacttttctactcctggcagaactaccgccgcggtagccttttttgcctttatccttgacaaatggagtcaagaaacccatttcagcagggattaccgtctggactgcttagcagtagctttgtggagaacatggaggtgccagcgcctgaatgcaatctccggctacttgccagtacagccggtagacacgctgaggatcctgagtctccagtcaccccaggaacaccaacgccgccagcagccgcagcaggagcagcagcaagaggaggaggaggaccgagaagagaacccgagagccggtctggaccctccggtggcggaggaggaggagtagctgacttgtttcccgagctgcgccgggtgctgactaggtcttccagtggacgggagagggggattaagcgggagaggcatgaggagactagccacagaactgaactgactgtcagtctgatgagccgcaggcgcccagaatcggtgtggtggcatgaggtgcagtcgcaggggatagatgaggtctcagtgatgcatgagaaatattccctagaacaagtcaagacttgttggttggagcccgaggatgattgggaggtagccatcaggaattatgccaagctggctctgaagccagacaagaagtacaagattaccaaactgattaatatcagaaattcctgctacatttcagggaatggggccgaggtggagatcagtacccaggagagggcggccttcagatgttgtatgatgaatatgtacccgggggtggtgggcatggagggagtcacctttatgaacacgaggttcaggggtgatgggtataatggggtggtctttatggccaacaccaagctgacagtgcacggatgctccttctttggcttcaataacatgtgcatcgaggcctggggcagtgtttcagtgaggggatgcagcttttcagccaactggatgggggtcgtgggcagaaccaagagcgtggtttcagtgaagaaatgtctgttcgagaggtgccacctgggggtgatgagcgagggcgaagccaaagtcaaacactgcgcctctaccgagacgggctgctttgtgctgatcaagggcaatgccaaagtcaagcataacatgatctgtggggcctcggatgagcgcggctaccagatgctgacctgcgccggtgggaacagccatatgctggccaccgtgcatgtggcctcgcacccccgcaagacatggcccgagttcgagcacaacgtcatgacccgatgcaacgtgcacctgggctcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttggggctgggtgggagtctgcatgatgggcagaatgactaaaatctgtgtttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcaaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgttgctgctgatggcccagctcgaggctctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgcccgcccaggttttccatgcactcatccatgatgatggcgatgggcccgtgggcggcggcctgggcaaagacgtttcgggggtcggacacatcatagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtgggaccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccaggggcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggtcggggtcaaaaacgaggtttcctccgtgctttttgatgcgtttcttacctctggtctccatgagttcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagcgccgcttgccagtcgcgcacggccagcgcgcgctcgtaggggctgaggggcatgccccagggcatggggtgcgtgagcgcagaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgcgccgaggttggagcgctgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcataaagttgcgagtgatgcggaaaggctggggcacctcggcccggttgttgatgacctgggcggcgagcacgatctcgtcgaagccgttgatgttgtggcccacgatgtagagttccacgaaccgtgggcggcccttgacgtggggcagcttcttgagctcctcgtaggtgagctcgtcagggtcgctgagcccgtgctgctcgagggcccagtcggcgagatggtggttggcgcggaggaaggaagtccagagatccacggccagggcggtttgcagacggtcccggtactgacggaactgctggccgacggccattttttcgggggtgacgcagtagaaggtgcgggggtccccgtgccagcgatcccatttgagctggagggcgagatcgagggcgagctcgacgaggcggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggagtgaccaccgtcccccgtttcttcttgggcgctgcttccatgccggtcagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggccccgggaacctcttcttccatctcctcttcttcctcttccactaacatctcttctacttcctcctcaggcggtggcgggggagggggcctgcgtcgccggcggcgcacgggcagacggtcgatgaagcgctcgatggtctcgccgcgccggcgtcgcatggtctcggtgacggcccgcccgtcctcgcggggccgcagcgtgaagacgccgccgcgcatttccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctggcgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccagatccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggccgggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgtttttgcaactttttttcggaggccggaaatgaagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagacccctagccagccgacttctccagttacggagcgagcccctcttttgttttttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctaaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgagcggtgagcgccagcaggcggcgcgagctgagcgaccaggagctgatgcacagcctgcagcgggccctgaccggggccgggaccgagggggagagctactttgacatgggcgcggacctgcactggcaacccagccgccgggccttggaggcggcggcaggaccctacgtagaagaggtggacgatgaggtggacgagggcgagtacctggaagactgatggcgcgaccgtatttttgctagatgcaacaacagccaccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagaccaacctggacaggatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagtatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacgcgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctaaacccccactggctaccgccgccggggttctacacgggcgagtacgacatgcccgaccccaatgacgggttcctgtgggacgatgtggacagcagcgtgttttccccccgaccgggtgctaacgagcgccccttgtggaagaaggaaggcagcgaccgacgcccgtcctcggcgctgtccggccgcgagggtgctgccgcggcggtgcccgaggccgccagtccttttcctagcttgcccttctcgctgaacagtattcgcagcagcgagctgggcaggatcacgcgcccgcgcttgctcggcgaggaggagtacttgaatgactcgctgttgagacccgagcgggagaagaacttccccaataacgggatagagagcctggtggacaagatgagccgctggaagacgtacgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtggtggtaacccgttcgctcacctgcgcccccgcatcgggcgcatgatgtaagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgaattcatgtacagcaacaagttcaaggcgcgggtcatggtctcccgcaagacccccaatggggtcaaagtagatgaaaattatgatggtagtcaggatgagctgaaatacgagtgggtggagtttgagctgcccgaaggcaacttctcggtgaccatgaccatcgacctgatgaacaacgccatcatcgacaattacttggcggtggggcggcagaacggggtcctggaaagcgacatcggcgtgaagttcgacactaggaacttcaggctgggctgggaccccgtgaccgagctggtcatgcccggggtgtacaccaacgaggccttccatcccgatgttgtcttgctgcccggctgcggggtggactttaccgagagccgcctcagcaacctgctgggcattcgcaagaggcagcccttccaggagggattccagatcatgtacgaggatctggaggggggcaacatccccgcgctcctggatgtcgaggcctatgaggaaagcaaggaaaaagctgaagccgaggcgactgcagccgtggctaccgccgcgaccaccaatgcagatgcaactaccaccagaggcgatacattcgccactgtggcggaggaagcagccgccctagcggtcgccgatgatagtgaaagtaagatagttatcaagccagtaaaagtggatagcaagaacagaagctacaacgtgctgccggacgaggtaaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggtcgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcagccagacgcgcggcctccggcagcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggtgaaggaggaaagaaagccccgcaaactgaagcgggtcaaaaaggacaaaaaggaggaggaagatgtggatggactggtggagtttgtgcgcgagttcgccccccggcggcgcgtgcagtggcgcgggcggaaagtgaagccggtgctgcggccaggcaccacggtggtcttcacgcccggcgagcgttccggctccgcctccaagcgctcctacgacgaggtgtacggggacgaggacatcctcgagcaggcggccgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgcccgcggcttccaccgccacccgccgaagacgcaagtacggcgcggccagcctgctgatgcccaactacgcgctgcatccttccatcatccccacgccgggctaccgcggcacgcgcttctaccgcggctacaccagccgccgccgcaagaccaccacccgccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaaagcatggatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagcagccagcccgcgaccctggacttgcctccgcctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgccnnnggaccgcgaggacaacacctactcgtacaaagtgcgctacacgctggccgtgggcgacanccgcgtgctggacatggccagcacntactttgacatccgcggcgtgctggatcggggccccagtttcaaaccntactccggcaccgcctacannngcctggctcccaagggagcgcccaacacctcacagtggaaggattccgacagcaaaatgcatacttttggagttgctgccatgcccggtgttgttggtaaaaaaatagaagccgatggtctgcctattggaatagattcatcctctggaactgataccataatttatgctgataaaactttccaaccagagccacaggttggaagtgacagttgggtcgacaccaatggtgcagaggaaaaatatggaggtagagctcttaaggacactacaaacatgaagccctgctacggttcttttgccaggcctaccaacaaagaaggtgggcaggctaacataaaagattctgaaactgccagcactactcctaactatgatatagatttggcattctttgacagcaaaaatattgccgctaactatgatccagatattgtaatgtacacagaaaatgttgagttgcaaactccagatactcatattgtgtttaagccaggaacttcagatgaaagttcagaagccaatttgggccagcaggccatgcccaacagacccaactacatcgggttcagagacaactttatcgggctcatgtactacaacagcactggcaatatgggtgtactggctggtcaggcctcccagctgaatgctgtggtggacttgcaggacagaaacaccgaactgtcctaccagctcttgcttgactctctgggtgacagaaccaggtatttcagtatgtggaatcaggcggtggacagctatgaccccgatgtgcgcattattgaaaatcacggtgtggaggatgaactccccaattattgcttccctttgaatggtgtgggctttacagatacttaccagggtgttaaagttaagacagatacagccgctgctggtaccaatggaacgcagtgggacaaagatgataccacagtcagcactgccaatgagatccactcaggcaatcctttcgccatggagatcaacatccaggccaacctgtggcggaacttcctctacgcgaacgtggcgctgtacctgcccgactcctacaagtacacgccggccaacatcacgctgccggccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggacagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgttgtccgagtgcaccagccccaccgcggcgtcatcgaggccgtctacctgcgcacgcccttctcggccggcaacgccaccacctaagccccgctcttgcttcttgcaagatgacggcctgtggctccggcgagcaggagctcagggccatcctccgcgacctgggctgcgggccctacttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgtctactttcgctcccaccgcgcgcgcatcgagaaggccaccgccttcgaccgcatgaatcaagacatgtaaactgtgtgtgtatgtgaatgctttattcatcataataaacagcacatgtttatgccaccttctctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttacggtacacggggttgcagcactggaacaccatcagggccgggtgcttcacgctcgccagcaccgtcgcgtcggtgatgccctccacgtccatatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagcccgcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcagccgtgcagccacagcgcgcagccggtgcactcccagttcttgtgggcgatctgggagtgcgagtgcacgaagccctgcaggaagcggcccatcatcgtggtcagggtcttgttgctggtgaaggtcagcggaatgccgcggtgctcctcgttcacatacaggtggcagatgcggcggtacacctcgccctgctcgggcatcagctggaaggcggacttcaggtcgctctccacgcggtaccggtccatcagcagcgtcatcacttccatgcccttctcccaggccgagacgatcggcaggctcagggggttcttcaccgttgtcatcttagtcgccgccgccgaggtcagggggtcgttctcgtccagggtctcaaacactcgcttgccgtccttctcgatgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcgtgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgcagccgacgaaaaccagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagcagcaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtgctcaagcgcacggtcgagctgacccacttcgcctacccggcgctcaacctgccccccaaggtcatgagcgccgtcatggaccaggtgctcatcaagcgcgcctcgcccctctccgaggacgagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgccgcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcactgccctcggacttcgtgccgctgaccttccgcgagtgccccccgccgctctggagccactgttacttgctgcgcctggccaactacctggcctaccactcggacgtgatcgaggacgtcagcggcgagggtctgctcgaatgccactgccgctgcaacctctgcacgccgcaccgctccctggcctgcaacccccagctgctgagcgaaacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacctggacccccagaccggagaggagctcaaccccagcttcccccaggatgccccgaggaagcagcaagaagctgaaagtggagctgccgctgccgccggaggatttggaggaagactgggagagcagtcaggcagaggaggaggagatggaagactgggacagcactcaggcagaggaggacagcctgcaagacagtctggaggaagacgaggtggaggaggaggcagaggaagaagcagccgccgccagaccgtcgtcctcggcggaggaggagaaagcaagcagcacggataccatctccgctccgggtcggggtcgcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagaaaaccagcggcagctagaaaatccacagcggcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacatacccgtctgaccctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgccgctccccgctactaacaaccaaactacccaccaacgccgccgtcgcgacctttctgaatctaatactaccacccacaccggaggtgagctccgaggtcgaccaacctctgggatttactacggcccctgggaggtggtagggttaatagcgctaggcctagttgcgggtgggcttttggctctctgctacctatacctcccttgctgttcgtacttagtggtgctgtgttgctggtttaagaaatggggaagatcaccctagtgagctgcggtgtgctggtggcggtggtggtgctttcgattgtgggactgggcggcgcggctgtagtgaaggaggagaaggccgatccctgcttgcatttcaatcccgacaaatgccagctgagttttcagcccgatggcaatcggtgcacggtgctgattaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcacacatgtgcgacacggtcatgtggatgagcaagcagtacgatatgtggccccccacgaaggagaacatcgtggtcttctccatcgcttacagcctgtgcacggcgctaatcaccgctatcgtgtgcctgagcattaacatgttcatcgctattcgccccagaaataatgccgaaaaagagaaacagccataacacgtttttttcacacacctttttcagaccatggcctctgttaaatttttgcttttatttgccagtctcattgccgtcattcatggaatgagtaatgagaaaattactatttacactggcactaatcacacattgaaaggtccagaaaaagccacagaagtttcatggtattgttattttaatgaatcagatgtatctactgaactctgtggaaacaataacaaaaaaaatgagagcattactctcatcaagtttcaatgtggatctgacttaaccctaattaacatcactagagactatgtaggtatgtattatggaactacagcaggcattttggacatggaattttatcaagtttctgtgtctgaacccaccacgcctagaatgaccacaaccacaaaaactacacctgttaccactatacagctcactaccaatggctttcttgccatgcttcaagtggctgaaaatagcaccagcattcaacccaccccacccagtgaggaaattcccagatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttaattttttctatcattacctctgctctatgcaattctgacaatgaggacgttactgtcgttgtcggatcaaattatacactgaaaggtccagcgaagggtatgctttcgtggtattgctattttggatctgacactacagaaactgaattatgcaatcttaagaatggcaaaattcaaaattctaaaattaacaattatatatgcaatggtactgatctgatactcctcaatatcacgaaatcatatgctggcagttacacctgccctggagatgatgctgacagtatgattttttacaaagtaactgttgttgatcccactactccacctccacccaccacaactactcacaccacacacacagatcaaaccgcagcagaggaggcagcaaagttagccttgcaggtccaagacagttcatttgttggcattacccctacacctgatcagcggtgtccggggttgctcgtcagcggtattgtcggtgtgctttcgggattagcagtcataatcatctgcatgttcatttttgcttgctgctatagaaggctttaccgacaaaaatcagacccactgctgaacctctatgtttaattttttccagagccatgaaggcagttagcgctctagttttttgttctttgattggcattgtttttagtgctgggtttttgaaaaatcttaccatttatgaaggtgagaatgccactctagtgggcatcagtggtcaaaatgtcagctggctaaaataccatctagatgggtggaaagacatttgcgattggaatgtcactgtgtatacatgtaatggagttaacctcaccattactaatgccacccaagatcagaatggtaggtttaagggccagagtttcactagaaataatgggtatgaatcccataacatgtttatctatgacgtcactgtcatcagaaatgagactgccaccacacagatgcccactacacacagttctaccactactaccatgcaaaccacacagacaaccactacatcaactcagcatatgaccaccactacagcagcaaagccaagtagcgcagcgcctcagccacaggctttggctttgaaagctgcgcaagctagtacaactactaggaccaatgagcagactactgattttttgtccactgtcgagagccacaccacagctacctcgagtgccttctctagcaccgccaatctctcctcgctttcctctacaccaatcagtcccgctactactcctagccccgctcatcttcccactcccctgaagcaaactgaggacagcggcatgcaatggcagatcaccctgctcattgtgatcgggttggtcatcctggccgtgttgctctactacatcttctgccgccgcattcccaacgcgcaccgcaagccggtctacaagcccatcattgtcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcatcacctgcatctgctgctgtagcatagtctgcctgcttatcaccttcttccagttcattgactggatctttgtgcgcatcgcctacctgcgccaccacccccagtaccacgaccagcgagtggcgcggctgctcaggctcctctgataagcatgcgggctctgctacttctcgcgcttctgctgttagtgctcccccgtcccgtcgacccccggtcccccactcagtcccccgaggaggttcgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcagcaacctcaggcacacgcactaccaccaccacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggacggcatagccatccaccagtgcaagaaaggcatcttctgcctggtgaaacaggccaagatctcctacgaggtcactccaaatgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgatttaaataaaaaaataatcatttgatttgaaataaagatacaatcatattgatgatttgagtttaacaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatcatcccttttacactaaagatggaaaattatccttacaagtttctccaccattaaatatactgagaacaagcattctaaacacactagctttaggttttggatcaggtttaggactccgtggctctgccttagcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaacggagatgtttcaaaacctatgcttctcactataaccctcaatggtactgatgacagcaacagtacatattcaatgtcattttcatacacctggactaatggaagctatgttggagcaacatttggggctaactcttataccttctcctacattgcccaagaatgaacactgtatcccaccctacattgcccaacccttcccaccccactctgtctatggaaaaaactctgaaacacaaaataaaataaagttcaagtgttttattgattcaacagttttacaggattcgagcagttatttttcctccaccctcccaagacatggaatacaccaccctctccccccgcacagccttgaacatttgaaagccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcaaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgtttaacagtccatagttcaacacgctccagccgaaactcatcgcgggaaggatgctacccacgtggccgtcgtaccagatcctcaggtaaatcaagtggcgctccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagggaagcgcgggtctcggtctcctcacagcgtggtaagggggccggccgatacgggtgatggcgggacgcggctgatcgtgttctcgaccgtgtcatgatgcagttgctttcggacattttcgtacttgctgtagcagaacctggtccgggcgctgcacaccgatcgccggcggcggtcccggcgcttggaacgctcggtgttgaagttgtaaaacagccactctcttagaccgtgcagcaaatctagggcctcaggagtgatgaagatcccatcatgcctgatagctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcaaaagcgggagggttctctaattcctcaatcatcatgttacactcctgcaccatccctagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcgtctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggcaagcaatttttaagaaaatcaacaaaagaaaaatcttccaggtgcacgtttagggcctcgggaacaacgatggagtacgtgcaaggggtgcgttccagcatggttagttagctgatctgtaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcggccgaggaagcaataaggcactacaatgctcagtctcaagtccagcaaagcgatgccatgcggatgaagcacaaaattctcaggtgcgtacaaaatgtaattactcccctcctgcacaggcagcgaagcccccgatccctccagatacacatacaaagcctcagcgtccatagcttaccgagcagcagcacacaacaggcgcaagagtcagagaaagactgagctctaacctgtccacccgctctctgctcaatatatagcccagatctacactgacgtaaaggccaaagtctaaaaatacccgccaaataatcacacacgcccagcacacgcccagaaaccggtgacacactcagaaaaatacgcgcacttcctcaaacgcccaaactgccgtcatttccgggttcccacgctacgtcatcagaattcgactttcaaattccgtcgaccgttaaaaatgtcacccgccccgcccctaacggtcgccgctcccacagccaatcacagccccgcatccccaaattcaaacagctcatttgcatattaacgcgcaccaaaagtttgaggtatattattgatgatg

SEQ ID NO: 4 - DNA, 40 bp, synthetic construct
  source 1..40: mol_type = other DNA
tctctatcactgatagggagatctctatcactgataggga

(19) United States
(12) Patent Application Publication
(10) Pub. No.: US 2025/0049912 A1
(43) Pub. Date: Feb. 13, 2025

(54) Multicistronic Vaccine and Methods for Producing and Using the Same

(71) Applicant: Ocugen, Inc., Malvern, PA (US)

(72) Inventors: Arun Kumar Upadhyay, Malvern, PA (US); Farokh Jal Dotiwala, Berwyn, PA (US); Dinesh Kumar Singh, Philadelphia, PA (US); Ilayaraja Muthuramu, Downingtown, PA (US)

(73) Assignee: Ocugen, Inc., Malvern, PA (US)

(21) Appl. No.: 18/586,220

(22) Filed: Feb. 23, 2024

Related U.S. Application Data
(60) Provisional application No. 63/447,734, filed on Feb. 23, 2023.

(51) Int. Cl. A61K 39/295; A61K 39/00; A61K 39/145; A61K 39/215; C12N 15/86

(52) U.S. Cl. CPC A61K 39/295; A61K 39/145; A61K 39/215; C12N 15/86; A61K 2039/5256; A61K 2039/543; A61K 2039/70; C12N 2710/10343; C12N 2760/16122; C12N 2760/16134; C12N 2760/16222; C12N 2760/16234; C12N 2770/20022; C12N 2770/20034

(57) ABSTRACT

The present disclosure provides multicistronic vaccines and method for producing and using the same in preventing infection or transmission, or reducing severity of disease caused by influenza and/or SARS-Cov-2 virus in a subject. Multicistronic vaccines of the disclosure can be administered via intramuscular, intranasal, or inhalation route. In one particular embodiments, the disclosure provides a recombinant adenovirus comprising at least two different extraneous oligonucleotides that are capable of stimulating an immune response in a subject. Each of the oligonucleotide independently comprises an oligonucleotide that encodes either influenza or SARS-Cov-2 virus antigens.

### CROSS-REFERENCE TO RELATED APPLICATIONS

This application claims the priority benefit of U.S. Provisional Application No. 63/447,734, filed Feb. 23, 2023, which is incorporated herein by reference in its entirety.

### REFERENCE TO AN ELECTRONIC SEQUENCE LISTING

The contents of the following electronic sequence listing is incorporated herein by reference in its entirety:
Name of the XML File: OCU-000900US_SEQ_LIST.xml;
File Size: 46,294 bytes;
Date of Creation: Feb. 23, 2024.

### FIELD OF THE DISCLOSURE

The present disclosure relates to multicistronic vaccines and method for producing and using the same in preventing infection or transmission, or reducing severity of disease caused by influenza and/or SARS-Cov-2 virus in a subject. Multicistronic vaccines of the disclosure can be administered via intramuscular, intranasal, or inhalation route.

### BACKGROUND OF THE DISCLOSURE

The COVID-19 pandemic caused by SARS-COV-2 continues to have a major impact on healthcare and social systems worldwide. Since COVID-19 and influenza share many clinical and epidemiological features, the optimal management of both respiratory diseases is paramount, particularly among healthcare workers and high-risk groups. Understanding the transmission dynamics between SARS-COV-2 and influenza viruses is essential to effectively prepare for their ongoing co-circulation.

The median basic reproduction number (R₀) for both of these viruses have been estimated to be 1.28 for influenza virus and 2.79 for SARS-COV-2 virus. Without being bound by any theory, it is believed that the difference in transmission potential (R₀) is likely due to the population immunity against influenza by the implementation of the national influenza vaccination policies, compared to the lack of pre-existing immunity to SARS-CoV-2. It is likely that during seasons with a severe influenza impact, COVID-19 is likely to exert an even more pronounced effect on healthcare systems. Therefore, holistic control measures are needed to mitigate the effects of both SARS-COV-2 and influenza viruses one of them being transmission control of both diseases by combined vaccination.

Furthermore, most of the Influenza and COVID-19 vaccines currently in use are designed for intramuscular (IM) immunization. Unfortunately, IM vaccines do not appear to provide a significant mucosal immunity, which is needed to prevent upper respiratory tract infection. The new Omicron variants depend less on cleavage by TMPRSS2 and more on nucleosome-mediated endocytosis for infecting cells. This reduces the latency period from 4-12 days to 3.5 days which is not sufficient for the activation of the immune memory response. Due to reduced latency period and mutations that escape antibody neutralization. Inhaled vaccines using updated (bi/multivalent) spike antigens from the latest Variants of Concern (VOC), produce not only a protective local humoral and cell-based immunity in the respiratory tract (IgA antibodies, CD4+, CD8+ T cells) but also a robust systemic immunity.

### SUMMARY OF THE DISCLOSURE

The present disclosure provides multicistronic vaccine and methods for producing and using the same. One particular aspect of the disclosure provides a recombinant adenovirus adapted for use in preventing infection or transmission, or reducing severity of disease caused by influenza and/or SARS-Cov-2 virus in a subject. The recombinant adenovirus comprises a two or more oligonucleotides that encode different immune response stimulating antigens in the subject. In particular, the recombinant adenovirus comprises at least two different oligonucleotides each of which is independently selected from the group consisting of:

(i) an oligonucleotide that encodes a hemagglutinin of influenza A virus or an immunogenic portion, variant, mutant, or fragment thereof;

(ii) an oligonucleotide that encodes a neuraminidase of influenza A virus or an immunogenic portion, variant, mutant, or fragment thereof;

(iii) an oligonucleotide that encodes a hemagglutinin of a first influenza B virus or an immunogenic portion, variant, mutant, or fragment thereof;

(iv) an oligonucleotide that encodes a hemagglutinin of a second influenza B virus or an immunogenic portion, variant, mutant, or fragment thereof, wherein said second influenza B virus is a different strain compared to said first influenza B virus;

(v) an oligonucleotide that encodes a spike protein (S-protein) or an immunogenic portion, variant, mutant, or fragment thereof of a first SARS-COV-2 virus; and

(vi) an oligonucleotide that encodes an S-protein or an immunogenic portion, variant, mutant, or fragment thereof of a second SARS-COV-2 virus, wherein said second SARS-COV-2 virus is a different strain compared to said first SARS-COV-2 virus;

and wherein an adenovirus of the recombinant adenovirus is selected from the group consisting of:

(i) a first, second, third, or fourth-generation human adenovirus 5;

(ii) a rare-serotype adenovirus;

(iii) a nonhuman adenovirus; and

(iv) a combination thereof.

It should be appreciated that oligonucleotides that encode an immune response stimulating antigen are different from one another, thereby allowing translation, transcription, and/or expression of different immune response stimulating antigens. In this manner, a single recombinant adenovirus, when transfected to a host, is able to express two or more immune response stimulating antigens within the host.

In some embodiments, said recombinant adenovirus is adapted for transfecting a host cell via intramuscular, intranasal, or inhalation route.

Yet in other embodiments, at least one of said oligonucleotides encodes at least one of 18 hemagglutinin (HA) subtypes 1-18 or an immunogenic portion, variant, mutant, or fragment thereof. In other embodiments, at least one of said oligonucleotides encodes at least one of 10 neuraminidase (NA) subtypes 1-10 or an immunogenic portion, variant, mutant, or fragment thereof. Still in other embodiments, at least one of said oligonucleotides encodes B/Yamagata/16/88 HA or an immunogenic portion, variant, mutant, or fragment thereof. In further embodiments, at least one of said oligonucleotides encodes B/Victoria/2/87 HA or an immunogenic portion, variant, mutant, or fragment thereof. Yet still in other embodiments, at least one of said oligonucleotides encodes said S-protein of said first SARS-COV-2 virus selected from the group consisting of SARS-COV-2 variants B.1.1.7, B.1.351, B1.1.28-P.1, B.1.617.2, B.1.1.529, BA.4, BA.5, BQ1.1, XBB.1.5, BA.2.75.2, and an immunogenic portion, variant, mutant, and fragment thereof. Still yet in other embodiments, at least one of said oligonucleotides encodes said S-protein of said second SARS-COV-2 virus.

In other embodiments, said recombinant adenovirus comprises at least three different oligonucleotides. Still in other embodiments, said recombinant adenovirus comprises at least four different oligonucleotides. Yet in other embodiments, said recombinant adenovirus comprises at least five different oligonucleotides. In further embodiments, said recombinant adenovirus comprises at least six different oligonucleotides.

In some embodiments, said rare-serotype adenovirus comprises Ad11, Ad26, Ad35, Ad48, Ad49, Ad50, or a combination thereof. Yet in other embodiments, said nonhuman adenovirus comprises simian Ad36, bovine Ad3, canine Ad2, porcine Ad3, or a combination thereof. In one particular embodiment, said nonhuman adenovirus comprises simian Ad36.

Still in other embodiments, the recombinant adenovirus further comprises 5′- and 3′-inverted terminal repeats (ITR) and a packaging signal (w). In other embodiments, the recombinant adenovirus further comprises (i) a promoter, (ii) an enhancer, (iii) a polyadenylation moiety, (iv) an internal ribosome entry site (IRES), (v) a self-cleaving protein site, or (vi) a combination thereof. In some instances, said self-cleaving protein site comprises T2A, P2A, E2A, F2A, or a combination thereof. In other instances, said polyadenylation moiety comprises simian virus 40 (SV40) polyadenylation (PolyA) moiety, bovine growth hormone (bGH) PolyA moiety, or a combination thereof.

Yet in other embodiments, the recombinant adenovirus further comprises a cytomegalovirus (CMV) promoter or enhancer, an elongation factor 1a (EF1a), a chicken β-actin (CBA) promoter, a CAG promotor, or a combination thereof.

Another aspect of the disclosure provides a plasmid comprising a genome of an adenovirus, wherein said genome of the adenovirus has been modified such that said genome comprises a transgene that is operatively linked to expression control sequences which direct transcription, translation, and/or expression in a host cell, and wherein said transgene comprises at least two different oligonucleotides, wherein each of said oligonucleotide is independently selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is a different strain than said first influenza B virus;
(v) an oligonucleotide that encodes a spike protein (S-protein) or an immunogenic portion, variant, mutant, or fragment thereof of a first SARS-COV-2 virus; and
(vi) an oligonucleotide that encodes an S-protein or an immunogenic portion, variant, mutant, or fragment thereof of a second SARS-COV-2 virus, wherein said second SARS-COV-2 virus is a different strain than said first SARS-COV-2 virus.

In some embodiments, said genome is derived from an adenovirus selected from the group consisting of:
(i) a first, second, third, or fourth-generation human adenovirus 5;
(ii) a rare-serotype adenovirus;
(iii) a nonhuman adenovirus; and
(iv) a combination thereof.

Still in other embodiments, said adenovirus is a nonhuman adenovirus. In some instances, said nonhuman adenovirus comprises simian Ad36, bovine Ad3, canine Ad2, porcine Ad3, or a combination thereof. Yet in other instances, said nonhuman adenovirus comprises simian Ad36. In one particular instance, wherein said genome of simian Ad36 lacks a native E1 and optionally, the E3 or E3B locus.

In further embodiments, at least on of said oligonucleotides encodes at least one of 18 hemagglutinin (HA) subtypes 1-18 or an immunogenic portion, variant, mutant, or fragment thereof. Yet in other embodiments, at least on of said oligonucleotides encodes at least one of 10 neuraminidase (NA) subtypes 1-10 or an immunogenic portion, variant, mutant, or fragment thereof. Still in other embodiments, at least on of said oligonucleotides encodes B/Yamagata/16/88 HA or an immunogenic portion, variant, mutant, or fragment thereof. In other embodiments, at least on of said oligonucleotides encodes B/Victoria/2/87 HA or an immunogenic portion, variant, mutant, or fragment thereof. Still in other embodiments, at least on of said oligonucleotides encodes said S-protein of said first SARS-COV-2 virus selected from the group consisting of SARS-COV-2 variants B.1.1.7, B.1.351, B1.1.28-P.1, B.1.617.2, B.1.1.529, BA.4, BA.5, BQ1.1, XBB. 1.5, BA.2.75.2, and an immunogenic portion, variant, mutant, and fragment thereof. Yet still in other embodiments, at least on of said oligonucleotides encodes said S-protein of said second SARS-COV-2 virus.

Yet in other embodiments, the plasmid comprises at least three different oligonucleotides. Still in other embodiments, said plasmid comprises at least four different oligonucleotides. In further embodiments, said plasmid comprises at least four different oligonucleotides. In other embodiments, said plasmid comprises at least five different oligonucleotides. Still yet in other embodiments, said plasmid comprises six different oligonucleotides.

Still another aspect of the disclosure provides a recombinant adenovirus (rAd) vector that comprises a genome of an adenovirus, wherein said genome of the adenovirus has been modified such that said genome comprises at least two different extraneous oligonucleotides that is operatively linked to expression control sequences which direct transcription, translation, and/or expression in a host cell, and wherein each of said extraneous oligonucleotide is independently selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is of a different strain than said first influenza B virus;
(v) an oligonucleotide that encodes a spike protein (S-protein) or an immunogenic portion, variant, mutant, or fragment thereof of a first SARS-COV-2 virus; and
(vi) an oligonucleotide that encodes an S-protein or an immunogenic portion, variant, mutant, or fragment thereof of a second SARS-COV-2 virus, wherein said second SARS-COV-2 virus is of a different strain than said first SARS-COV-2 virus.

In some embodiment, an adenovirus of said recombinant adenovirus is selected from the group consisting of:
(i) a first, second, third, or fourth-generation human adenovirus 5;
(ii) a rare-serotype adenovirus;
(iii) a nonhuman adenovirus; and
(iv) a combination thereof.

Yet in other embodiments, the rAd vector further comprises naturally occurring adenovirus (Ad) major serotype capsid proteins (hexon, penton base, and fiber) and four minor proteins (IIIa, VI, VIII, and IX). In other embodiments, said rAd vector is adapted for transfecting a host cell via intramuscular, intranasal, or inhalation route. Still in other embodiments, said nonhuman adenovirus comprises simian Ad36, bovine Ad3, canine Ad2, porcine Ad3, or a combination thereof. In some instances, said nonhuman adenovirus comprises simian Ad36.

Still in other embodiments, the rAd vector includes 5′- and 3′-inverted terminal repeats (ITR) and a packaging signal (v). In other embodiments, the rAd vector further comprises (i) a promoter, (ii) an enhancer, (iii) a polyadenylation moiety, (iv) an internal ribosome entry site (IRES), (v) a self-cleaving protein site, or (vi) a combination thereof. In some instances, said self-cleaving protein site comprises T2A, P2A, E2A, F2A, or a combination thereof. Yet in other instances, said polyadenylation moiety comprises simian virus 40 (SV40) polyadenylation (PolyA) moiety, bovine growth hormone (bGH) PolyA moiety, or a combination thereof.

In further embodiments, the rAd vector also includes a cytomegalovirus (CMV) promoter or enhancer, an elongation factor 1a (EF1a), a chicken β-actin (CBA) promoter, a CAG promotor, or a combination thereof.

In some embodiments, at least one of said oligonucleotides encodes at least one of 18 hemagglutinin (HA) subtypes 1-18 or an immunogenic portion, variant, mutant, or fragment thereof. In other embodiments, at least one of said oligonucleotides encodes at least one of 10 neuraminidase (NA) subtypes 1-10 or an immunogenic portion, variant, mutant, or fragment thereof. Still in other embodiments, at least one of said oligonucleotides encodes B/Yamagata/16/88 HA or an immunogenic portion, variant, mutant, or fragment thereof. Yet in other embodiments, at least one of said oligonucleotides encodes B/Victoria/2/87 HA or an immunogenic portion, variant, mutant, or fragment thereof. In further embodiments, at least one of said oligonucleotides encodes said S-protein of said first SARS-CoV-2 virus selected from the group consisting of SARS-COV-2 variants B.1.1.7, B.1.351, B1.1.28-P.1, B.1.617.2, B.1.1.529, BA.4, BA.5, BQ1.1, XBB.1.5, BA.2.75.2, and an immunogenic portion, variant, mutant, and fragment thereof. Still yet in other embodiments, at least one of said oligonucleotides encodes said S-protein of said second SARS-COV-2 virus.

Still in other embodiments, said recombinant adenovirus comprises at least three different oligonucleotides. Yet in other embodiments, said recombinant adenovirus comprises at least four different oligonucleotides. In other embodiments, said recombinant adenovirus comprises at least four different oligonucleotides. In further embodiments, said recombinant adenovirus comprises at least five different oligonucleotides. In yet other embodiments, said recombinant adenovirus comprises six different oligonucleotides.

Yet in other embodiments, said rare-serotype adenovirus comprises Ad11, Ad26, Ad35, Ad48, Ad49, Ad50, or a combination thereof.

Another aspect of the disclosure provides a pharmaceutical composition comprising any one of the recombinant adenovirus (rAd) vector disclosed herein and a pharmaceutically acceptable excipient.

Still another aspect of the disclosure provides a method for administering a flu or SARS-COV-2 virus vaccine, wherein said vaccine is a polyvalent vaccine comprising at least two different extraneous oligonucleotides, said method comprising administering a therapeutically effective amount of a recombinant adenovirus (rAd) vector to a subject in need of such a vaccine, thereby eliciting an immune response in said subject, wherein said rAd vector comprises a genome of an adenovirus, and wherein said genome of the adenovirus has been modified such that said genome comprises at least two different extraneous oligonucleotides that is operatively linked to expression control sequences which direct transcription, translation, and/or expression in a host cell, and wherein each of said extraneous oligonucleotide is independently selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is of a different strain than said first influenza B virus;
(v) an oligonucleotide that encodes a spike protein (S-protein) or an immunogenic portion, variant, mutant, or fragment thereof of a first SARS-COV-2 virus; and
(vi) an oligonucleotide that encodes an S-protein or an immunogenic portion, variant, mutant, or fragment thereof of a second SARS-COV-2 virus, wherein said second SARS-COV-2 virus is of a different strain than said first SARS-COV-2 virus.

In some embodiments, said vaccine is administered to said subject via intramuscular, intranasal, or inhalation route. Yet in other embodiments, said vaccine is a bivalent vaccine. Still in other embodiments, said vaccine is a trivalent vaccine. In further embodiments, said vaccine is a quadrivalent vaccine. In further embodiments, said vaccine is a pentavalent vaccine. Yet in other embodiments, said vaccine is a hexavalent vaccine.

Still in other embodiments, an adenovirus of said rAd vector is selected from the group consisting of:
(i) a first, second, third, or fourth-generation human adenovirus 5;
(ii) a rare-serotype adenovirus;
(iii) a nonhuman adenovirus; and
(iv) a combination thereof.

Yet in other embodiments, said nonhuman adenovirus comprises simian Ad36, bovine Ad3, canine Ad2, porcine Ad3, or a combination thereof. In other embodiments, said nonhuman adenovirus comprises simian Ad36.

Further aspect of the disclosure provides a method for reducing or preventing an incidence of or reducing a severity of influenza or SARS-COV-2 infection, said method comprising administering a therapeutically effective amount of any one of a recombinant adenovirus disclosed herein or any one of a recombinant adenovirus (rAd) vector disclosed herein or any pharmaceutical composition disclosed herein.

One particular aspect of the disclosure provides a method for reducing or preventing an incidence of or reducing a severity of influenza virus infection, SARS-CoV-2 virus infection, or a combination thereof in a subject. The method includes administering a therapeutically effective amount of a polycistronic vaccine to the subject, wherein said polycistronic vaccine comprises a recombinant adenovirus (rAd) vector or a pharmaceutical composition comprising said rAd vector. The rAd vector can be any one of the rAd vector disclosed herein. In one particular embodiment, the rAd vector comprises a genome of an adenovirus that has been modified such that said genome comprises (i) a first extraneous oligonucleotide encoding at least one immune response stimulating antigen of an influenza virus and (ii) a second extraneous oligonucleotide encoding at least one immune response stimulating antigen of a SARS-COV-2 virus. In some embodiments, said first extraneous oligonucleotide is selected from the group consisting of: (i) an oligonucleotide that encodes a hemagglutinin of influenza A virus or an immunogenic portion, variant, mutant, or fragment thereof; (ii) an oligonucleotide that encodes a neuraminidase of influenza A virus or an immunogenic portion, variant, mutant, or fragment thereof; (iii) an oligonucleotide that encodes a hemagglutinin of a first influenza B virus or an immunogenic portion, variant, mutant, or fragment thereof; (iv) an oligonucleotide that encodes a hemagglutinin of a second influenza B virus or an immunogenic portion, variant, mutant, or fragment thereof, wherein said second influenza B virus is of a different strain than said first influenza B virus; and (v) a combination of two of more said oligonucleotides of (i)-(iv). Yet in other embodiments, said second extraneous oligonucleotide is selected from the group consisting of: (vi) an oligonucleotide that encodes a spike protein (S-protein) or an immunogenic portion, variant, mutant, or fragment thereof of a first SARS-COV-2 virus; (vii) an oligonucleotide that encodes an S-protein or an immunogenic portion, variant, mutant, or fragment thereof of a second SARS-COV-2 virus, wherein said second SARS-COV-2 virus is of a different strain than said first SARS-COV-2 virus; and (viii) a combination of said oligonucleotides of (vi) and (vii).

Still in some embodiments, the vaccine is administered intranasally.

### BRIEF DESCRIPTION OF THE DRAWINGS

FIG. 1 is a schematic illustration showing hemagglutinin (HA) and neuraminidase (NA) proteins on the surface of influenza virus and spike proteins on the surface of SARS-COV-2 virus.

FIG. 2 is prior art diagram of transgene cassettes for SARS-COV-2 vaccine: ChAd-control has no transgene insert; ChAd-SARS-COV-2-S encodes for SARS-COV-2 S-protein with the two indicated proline mutations. See, WO 2021/247567, published Dec. 9, 2021.

FIG. 3 is a schematic illustration of the wild-type Human Ad5 vector that is used in producing rAd vectors of the disclosure.

FIG. 4 is a schematic illustration of the first generation Ad5 vector that is used in producing rAd vectors of the disclosure.

FIG. 5 is a schematic illustration of the second generation Ad5 vector that is used in producing rAd vectors of the disclosure.

FIG. 6 is a schematic illustration of the third generation Ad5 vector that is used in producing rAd vectors of the disclosure.

FIG. 7 is a schematic illustration of the fourth generation Ad5 vector that is used in producing rAd vectors of the disclosure.

FIG. 8 is a schematic illustration showing the maximum transgene carrying capacity of 1^{st}-4^{th} generation human Ad5 vectors.

FIG. 9 is a schematic illustration showing comparison of maximal transgene capacity of gutted human Ad5 vector compared to the first and the second generation human Ad5 vectors.

### DESCRIPTION OF THE DISCLOSURE

The present disclosure is based, at least in part, on the development of recombinant non-human adenoviral vector compositions and immunogenic compositions thereof for treating or preventing coronavirus (COVID-19) and/or flu infections. In addition, the disclosure provides methods of administering the compositions disclosed herein. Compositions disclosed herein provide immunity against a coronavirus and/or flu infections in a single dosage unit. In one particular embodiment, the present disclosure provides compositions for administering the vaccine via intramuscular, intranasal, or inhalation route. Compositions of the disclosure can be used in preventing infection or transmission, or reducing severity of disease caused by influenza and/or SARS-Cov-2 virus in a subject.

The term “antibody” refers to an immunoglobulin, antigen-binding fragment, or derivative thereof, which specifically binds and recognizes an analyte (antigen) such as a coronavirus S protein, an antigenic fragment thereof, or a dimer or multimer of the antigen. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SFI, F(ab′)2; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

“Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The viral genome is capped, polyadenylated, and covered with nucleocapsid proteins. The coronavirus virion includes a viral envelope containing type I fusion glycoproteins referred to as the spike(S) protein (S-protein). Most coronaviruses have a common genome organization with the replicase gene included in the 5′-portion of the genome, and structural genes included in the 3′-portion of the genome.

Coronavirus Spike(S) protein: A class I fusion glycoprotein initially synthesized as a precursor protein. Individual precursor S polypeptides form a homotrimer and undergo glycosylation within the Golgi apparatus as well as processing to remove the signal peptide, and cleavage by a cellular protease to generate separate S1 and S2 polypeptide chains, which remain associated as S1/S2 protomers within the homotrimer and is therefore a trimer of heterodimers. The S1 subunit is distal to the virus membrane and contains the receptor-binding domain (RBD) that mediates virus attachment to its host receptor. The S2 subunit contains fusion protein machinery, such as the fusion peptide, two heptad-repeat sequences (HR1 and HR2) and a central helix typical of fusion glycoproteins, a transmembrane domain, and the cytosolic tail domain.

Coronavirus Spike(S) protein prefusion conformation is a structural conformation adopted by the ectodomain of the coronavirus S protein following processing into a mature coronavirus S protein in the secretory system, and prior to triggering of the fusogenic event that leads to transition of coronavirus S to the post-fusion conformation. The three-dimensional structure of an exemplary coronavirus S protein (HKU1-CoV) in a prefusion conformation is disclosed herein and provided in Kirchdoerfer et al., “Pre-fusion structure of a human coronavirus spike protein,” Nature, 531:118-121, 2016 (incorporated by reference herein).

A coronavirus S ectodomain trimer “stabilized in a prefusion conformation” comprises one or more amino acid substitutions, deletions, or insertions compared to a native coronavirus S sequence that provide for increased retention of the prefusion conformation compared to coronavirus S ectodomain trimers formed from a corresponding native coronavirus S sequence. The “stabilization” of the prefusion conformation by the one or more amino acid substitutions, deletions, or insertions can be, for example, energetic stabilization (for example, reducing the energy of the prefusion conformation relative to the post-fusion open conformation) and/or kinetic stabilization (for example, reducing the rate of transition from the prefusion conformation to the post-fusion conformation). Additionally, stabilization of the coronavirus S ectodomain trimer in the prefusion conformation can include an increase in resistance to denaturation compared to a corresponding native coronavirus S sequence. Methods of determining if a coronavirus S ectodomain trimer is in the prefusion conformation include, but are not limited to, negative-stain electron microscopy and antibody binding assays using a prefusion-conformation-specific antibody.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

In one example, a desired response is to inhibit or reduce or prevent influenza and/or SARS-COV-2 infection. The infection does not need to be completely eliminated or reduced or prevented for the method to be effective. For example, administration of an effective amount of the immunogen can induce an immune response that decreases the infection (for example, as measured by infection of cells, or by number or percentage of subjects infected) by a desired amount, for example by at least 50%, at least 60%, at least 70%, at least 80%, at least 90%, at least 95%, at least 98%, or even at least 100% (elimination or prevention of detectable infection), as compared to a suitable control.

Expression refers to transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences refer to nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′- or 3′-regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “heterologous” refers to originating from a different genetic source. A nucleic acid molecule that is heterologous to a cell originated from a genetic source other than the cell in which it is expressed. In one specific, non-limiting example, a heterologous nucleic acid molecule encoding a recombinant coronavirus S protein is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cells refer to cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

An Immune response is a response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

A nucleic acid molecule is a polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

The term “operably linked” refers to a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

A polypeptide is any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

A prime-boost vaccination is an immunotherapy including administration of a first immunogenic composition (the primary vaccine) followed by administration of a second immunogenic composition (the booster vaccine) to a subject to induce an immune response. The priming vaccine and/or the booster vaccine include a vector (such as a viral vector, RNA, or DNA vector) expressing the antigen to which the immune response is directed. The booster vaccine is administered to the subject after the priming vaccine; a suitable time interval between administration of the priming vaccine and the booster vaccine, and examples of such timeframes are disclosed herein. In some embodiments, the priming vaccine, the booster vaccine, or both primer vaccine and the booster vaccine additionally include an adjuvant. In one non-limiting example, the priming vaccine is a DNA-based vaccine (or other vaccine based on gene delivery), and the booster vaccine is a protein subunit or protein nanoparticle based vaccine.

A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant virus is one that includes a genome that includes a recombinant nucleic acid molecule. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity is the similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods. Methods of alignment of sequences for comparison are well known in the art. Various programs and alignment algorithms are described in: Smith & Waterman, Adv. Appl. Math. 2:482, 1981; Needleman & Wunsch/. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al, Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al, J. Mol. Biol. 215:403-10, 1990, presents a detailed consideration of sequence alignment methods and homology calculations.

Homologs and variants of a polypeptide (such as a coronavirus S ectodomain) are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. As used herein, reference to “at least 90% identity” or similar language refers to “at least 90%, at least 91%, at least 92%, at least 93%, at least 94%, at least 95%, at least 96%, at least 97%, at least 98%, at least 99%, or even 100% identity” to a specified reference sequence.

A vaccine is a pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In a non-limiting example, a vaccine induces an immune response that reduces the severity of the symptoms associated with SARS-COV-2 virus and/or influenza virus infection and/or decreases the viral load compared to a control. In another non-limiting example, a vaccine induces an immune response that reduces and/or prevents SARS-COV-2 virus and/or influenza virus infection compared to a control.

A vector is an entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

Virus-like particle (VLP) are a non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994)/. Virol. 68:4503-4505; Vincente, J Invertebr Pathol., 2011; Schneider-Ohrum and Ross, Curr. Top. Microbiol. Immunol, 354:53073, 2012).

A composition of the present disclosure may comprise one or more active agents. In some embodiments, an active agent may be an agent to prevent, treat, or reduce the infectivity of influenza and/or SARS-COV-2 viral infection. In some embodiments, treating a viral infection may comprise reducing the infectivity and/or transmission of the virus. In some embodiments, preventing a viral infection may comprise reducing the infectivity and/or transmission of the virus. A composition of the present disclosure may comprise an active agent to prevent a viral infection, an active agent to treat a viral infection, an active agent to reduce the infectivity of a viral infection, or a combination thereof. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, sweeteners, colorants, odorants, salts (substances of the present disclosure may themselves be provided in the form of a pharmaceutically acceptable salt), buffers, coating agents, or antioxidants.

The present disclosure relates to non-human adenoviral vector compositions and methods of using an immunogenic composition comprising the adenovirus vector and optionally one or more additional active ingredients, a pharmaceutically acceptable carrier, diluent, excipient or adjuvant to treat or prevent a respiratory viral infection. Using a simian adenovirus vector in some embodiments can overcome issues of heterologous vector cross-immunity that has been seen with human adenovirus vector platforms (PMID: 32450106).

In one particular aspect of the disclosure, an adenoviral vector is provided. Adenoviruses are non-enveloped viruses comprising a nucleocapsid and a linear double stranded DNA genome. The viral nucleocapsid comprises penton and hexon capsomers. A unique fiber is associated with each penton base and aids in the attachment of the virus to the host cell via the Coxsackie-adenovirus receptor on the surface of the host cell. The genome of adenoviruses comprises 4 early transcriptional units (E1, E2, E3 and E4), which have mainly regulatory functions and prepare the host cell for viral replication. The genome also comprises 5 late transcriptional units (L1, L2, L3, L4 and L5), which encode structural proteins including the penton (L2), the hexon (L3), the scaffolding protein (L4) and the fiber protein (L5), which are under the control of a single promoter. Each extremity of the genome comprises an Inverted Terminal Repeat (ITR) which is necessary for viral replication.

Adenoviruses offer many other advantages for clinical vaccine development. The adenoviral genome is relatively small, well characterized and easy to manipulate. The deletion of a single transcriptional unit, E1, renders the virus replication-incompetent which increases its predictability and reduces side effects in clinical applications. Recombinant adenoviruses can accommodate relatively large transgenes allowing flexibility in subunit design, and have a relatively broad tropism facilitating transgene delivery to a wide variety of cells and tissues. Importantly for clinical applications, methods for scaled-up production and purification of recombinant adenoviruses to high titre are well established. Thus far, subgroup C serotypes AdHu2 or AdHu5 have predominantly been used as vectors. However, the first generation of vaccine vectors based on the archetypal human adenovirus AdHu5 showed poor efficacy in clinical trials, despite encouraging preclinical data. It was subsequently discovered that a large proportion of human adults harbor significant titres of neutralizing antibodies to common human serotypes such as AdHu2 and AdHu5, as a result of natural infection. Neutralizing antibodies could reduce the potency of viral vector vaccines by blocking viral entry into host cells and hence delivery of the target transgene.

The compositions described herein include vectors that deliver a heterologous molecule to cells, either for therapeutic or vaccine purposes. As used herein, a vector may include any genetic element including, without limitation, naked DNA, a phage, transposon, cosmid, episome, plasmid, or a virus. In some embodiments such vectors contain simian adenovirus DNA (e.g., SAdV-36), and a transgene. By “transgene” is meant the combination of a selected heterologous gene and the other regulatory elements necessary to drive translation, transcription and/or expression of the gene product in a host cell. In several embodiments, the viral vector can include an adenoviral vector that expresses a transgene encoding a coronavirus S protein. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 36, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 36.

Typically, a SAdV-derived adenoviral vector is designed such that the transgene is located in a nucleic acid molecule which contains other adenoviral sequences in the region native to a selected adenoviral gene. The transgene may be inserted into an existing gene region to disrupt the function of that region, if desired. Alternatively, the transgene may be inserted into the site of a partially or fully deleted adenoviral gene. For example, the transgene may be located in the site of such as the site of a functional E1 deletion or functional E3 deletion (or E3B), among others that may be selected. The term “functionally deleted” or “functional deletion” means that a sufficient amount of the gene region is removed or otherwise damaged, e.g., by mutation or modification, so that the gene region is no longer capable of producing functional products of gene expression. If desired, the entire gene region may be removed. In one embodiment, the adenovirus vector useful according to the disclosure is a simian adenovirus, SAd36, having deletions in the E1 and E3B genes. In one aspect, the adenovirus vector has the nucleic acid sequence of SEQ ID NO: 3. In another aspect, the adenoviral vector has the nucleic acid sequence with at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity with SEQ ID NO: 3.

For example, for a production vector useful for generation of a recombinant virus, the vector may contain the transgene and either the 5′-end of the adenoviral genome or the 3′-end of the adenoviral genome, or both the 5′- and 3′-ends of the adenoviral genome. The 5′-end of the adenoviral genome contains the 5′ cis-elements necessary for packaging and replication; i.e., the 5′-inverted terminal repeat (ITR) sequences (which function as origins of replication) and the native 5′ packaging enhancer domains (that contain sequences necessary for packaging linear Ad genomes and enhancer elements for the E1 promoter). The 3′-end of the adenoviral genome includes the 3′ cis-elements (including the ITRs) necessary for packaging and encapsidation. Suitably, a recombinant adenovirus contains both 5′- and 3′-adenoviral cis-elements and the transgene is located between the 5′- and 3′-adenoviral sequences. A simian based adenoviral vector (e.g., SAdV-36) may also contain additional adenoviral sequences.

Suitably, these simian based adenoviral vectors contain one or more adenoviral elements derived from the adenoviral genome. In one embodiment, the vectors contain adenoviral sequences that are derived from a different adenoviral serotype than that which provides the ITRs. As defined herein, a pseudotyped adenovirus refers to an adenovirus in which the capsid protein of the adenovirus is from a different adenovirus than the adenovirus which provides the ITRs. Chimeric or hybrid adenoviruses may be constructed using the adenoviruses described herein using techniques known to those of skill in the art. See, e.g., U.S. Pat. No. 7,291,498.

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell.

SARS-COV-2, like that of SARS-COV-1 and Middle East respiratory syndrome coronavirus (MERS-COV) that caused MERS, binds to angiotensin converting enzyme 2 (ACE2) on the surface of the cells in order to infect the cells. Briefly, ACE-2 is the functional receptor for SARS-COV-1, SARS-COV-2, and MERS-COV and most likely future SARS-COV variants. ACE-2 is an important component of Renin-Angiotensin-Aldosterone System (RAAS). ACE-2 converts angiotensin 2 to angiotensin 1-7. High angiotensin 2 is associated with vasoconstriction, inflammation, and acute lung injury. ACE2 is expressed in various organs including lungs, heart, kidney, liver, intestine, and other tissues. SARS-COV virus binds to ACE-2 and enter the cells.

The SARS-COV-2 RNA genome is approximately 30,000 nucleotides in length. The 5′ two-thirds encode nonstructural proteins that enable genome replication and viral RNA synthesis. The remaining one-third encode structural proteins such as spike(S), envelope, membrane, and nucleoprotein (NP) that form the spherical virion, and accessory proteins that regulate cellular responses. The S-protein forms homotrimeric spikes on the virion and engages the cell-surface receptor angiotensin-converting enzyme 2 (ACE2) to promote coronavirus entry into human cells. S-proteins are cleaved sequentially during the entry process to yield S1 and S2 fragments, followed by further processing of S2 to yield a smaller S2′ protein. The S1 protein includes the receptor binding domain (RBD) and the S2 protein promotes membrane fusion. The structure of a soluble, stabilized prefusion form of the SARS-COV-2 S protein was solved by cryo-electron microscopy, revealing considerable similarity to the SARS-CoV S-protein. This form of the S-protein has been used as vaccine target.

In virus classification, influenza viruses are negative sense, single strand RNA viruses. The genera of influenza viruses currently comprise the Orthomyxoviridae Family: Influenza virus A, Influenza virus B, and Influenza virus C. Each of these genera contains a single species of influenza virus. The genus Influenza virus A consists of a single species, influenza A virus, which includes all of the influenza virus strains currently circulating among humans.

The different types of Influenza A are classified based on two proteins on the surface of the virus: hemagglutinin (H) and neuraminidase (N). There are at least 18 different hemagglutinin subtypes and at least 11 different neuraminidase subtypes (H1 through H18 and N1 through N11, respectively). These include, for example, but not limited to, H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7 serotypes. Influenza A subtypes (e.g., H1N1) can be further broken down into different genetic clades (e.g., 6B.1) and sub-clades (e.g., 6B.1A). Although clades and sub-clades are genetically different they are not necessarily antigenically different. There are potentially many more influenza A subtype combinations given the propensity for virus reassortment. Reassortment is a process by which influenza viruses swap gene segments. Reassortment can occur when two different influenza viruses infect the same host at the same time and swap genetic information.

The genus Influenza virus B consists of a single species, influenza B virus, of which there is currently only one known serotype, but instead are further classified into two lineages: B/Yamagata and B/Victoria. Influenza B virus is almost exclusively a human pathogen but is significantly less common and less genetically diverse than influenza A strains. Because of this limited genetic diversity, most humans acquire a certain degree of immunity to influenza B virus at an early age; however, the mutation frequency of the virus is sufficiently high enough to prevent lasting immunity by most humans, but not high enough to permit pandemic infection by influenza B virus across human populations. Similar to influenza A viruses, influenza B viruses can then be further classified into specific clades (e.g., V1A) and sub-clades (e.g., V1A1).

The genus Influenza virus C also consists of a single species, denoted influenza C virus, of which there is also currently only one known serotype. This serotype is known to infect both primates and porcine, and while human infections of influenza C virus are rare, the resulting illness can be mild to severe. Epidemics of influenza C virus are not uncommon in exposed populations, however, due to its rapid transmissibility in humans having close contact.

A fourth family of influenza viruses—Influenza D—was first isolated in 2011 and identified in 2016.

Hemagglutinin (HA) and neuraminidase (NA) are the two large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins are targets for antiviral drugs. Furthermore, they are antigens to which antibodies can be raised. Influenza A viruses are classified into subtypes based on antibody responses to HA and NA. These different types of HA and NA form the basis of the H and N distinctions in, for example, H5N1. There are 18 HA and 11 NA subtypes known, but only HA 1, 2 and 3, and NA 1 and 2 are commonly found in humans. Influenza A virus, in particular, has many different serotypes, upwards of 144 possible “HN” serotypes based on variations within these two proteins alone. Only a small number of these combinations are believed to be circulating within susceptible populations at any given time.

Influenza viruses are etiologic agents for a contagious respiratory illness (commonly referred to as the flu) that primarily affects humans and other vertebrates. Influenza is highly infectious and an acute respiratory disease that has plagued the human race since ancient times. Infection is characterized by recurrent annual epidemics and periodic major worldwide pandemics. Influenza virus infection can cause mild to severe illness and can even lead to death. Every year in the United States, 5 to 20 percent of the population, on average, contracts the flu with more than 200,000 hospitalizations from complications and over 36,000 deaths. Because of the high disease-related morbidity and mortality, direct and indirect social economic impacts of influenza are enormous. Four pandemics occurred in the last century, together causing tens of millions of deaths worldwide.

In the case of influenza, the immunodominant HA and NA proteins protrude from the central capsid of the viral particle, and so they tend to interact most strongly with the host's internal environment and dominate the host immune response. Mutations occurring in the microbial genome that protect the microbe from the host immune system, these mutations are most readily found to affect the immunodominant antigens.

Non-immunodominant antigens are those that are capable of raising a host immune response but account for only a small amount of the total immune response. This is thought to happen because the non-immunodominant antigens are at least partially shielded from the host immune system, as in the case of an antigen that is located in a cleft or fold of the microbial surface or is surrounded by protruding elements of the microbe. In the case of influenza, non-immunodominant antigens occurring near the capsid surface are shielded from the host immune system by the immunodominant HA and NA spikes protruding from the surface. Non-immunodominant antigens tend to show less mutation in response to host immune pressure than do immunodominant antigens.

The CDC and the leading authorities on disease prevention in the world recommend the single best way of preventing a viral respiratory infection, such as SARS and flu, in humans is through regular vaccinations. Conventional vaccines typically target a single virus. Thus, a separate vaccine is administered for influenza virus, and SARS-COV-2 virus. Separate dosage leads to decreased compliance, and sometimes require a “rest period” between administration of vaccines for influenza virus and SARS-COV-2 virus.

As for flu vaccine, conventional vaccines target the immunodominant proteins, HA and NA antigens for influenza. These vaccines have not been universally protective or 100 percent effective at preventing the disease. Antigenic shift prevents flu vaccines from being universally protective or from maintaining effectiveness over many years. The ineffectiveness of conventional vaccines may also be due, in part, to antigenic drift and the resulting variation within antigenic portions of the HA and NA proteins most commonly recognized by the immune system (i.e., immunodominant antigens). As a result, many humans may find themselves susceptible to the flu virus without an effective method of treatment available since influenza is constantly improving its resistant to current treatments. This scenario is particularly concerning with respect to the H5N1 virus, which is highly virulent but for which there is currently no widely available commercial vaccine to immunize susceptible human populations.

Some aspects of the disclosure provide multicistronic (i.e., multivalent) vaccine and methods for producing and using the same. In this manner, compositions of the disclosure can be used for vaccination against influenza virus and SARS-CoV-2 virus. Alternatively, compositions of the disclosure can be targeted to two or more different strains of SARS-COV-2 virus, two or more different strains of influenza virus, or a combination of SARS. In one particular embodiment, compositions disclosed herein include at least one oligonucleotide that encodes immune response stimulating antigen of SARS-COV-2 virus and at least one oligonucleotide that encodes immune response stimulating antigen of influenza virus. In this manner, some compositions of the disclosure provide concomitant immunization or protection against both SARS-COV-2 virus and influenza virus.

Antigens and epitopes (i.e., immune stimulating antigens or immune response stimulating antigens) as disclosed herein contain or are derived from a plurality of antigenic regions (e.g., epitopes) of a pathogen or of different pathogens, such as influenza virus and SARS-COV-2 virus. Composite antigens of the disclosure may contain an antigenic region that represents a combination of all or parts of two or more similar or dissimilar epitopes, or a plurality of immunologically responsive regions derived from one or multiple antigenic sources (e.g., epitopes of virus particles). These immunological regions are amino acid sequences or epitopes that are representative of sequences found at those antigenic regions of a pathogen or other antigen associated with an infection or a disease or, importantly, associated with stimulation of the immune system to provide protection against the pathogen. Administration may be to individuals such as via injection (e.g., intradermal, intradermal, intravenous, oral, nasal, intraperitoneal).

Some embodiments of the disclosure are directed to antigens of a pathogen, such as influenza virus or SARS-COV-2 virus antigens. As used herein, unless the context requires otherwise the terms “antigen” and “epitope” are used interchangeably herein and refer to an immune response stimulating antigen that is produced or expressed by the oligonucleotide used in the vaccine composition of the disclosure. Antigens may be selected regions of the virus (e.g., S-protein of SARS-COV-2 virus) that are known or believed to generate an effective immune response after administration. The peptide sequence of the antigen may contain a plurality of immunologically responsive regions or epitopes of one or more pathogens, which are artificially arranged (i.e., not naturally occurring), typically along a single amino acid sequence or peptide. The plurality may contain multiples of the same pathogen (e.g., different epitopes of influenza virus or different S-proteins of SARS-COV-2 virus), although generally not in a naturally occurring order, or typically multiples of a variety of different epitopes from one or more different viruses or different strains of viruses. Epitopes may be identical to known immunological regions of a pathogen, or entirely new constructs that have not previously existed and therefore artificially constructed. Preferably, antigens generated by the vaccine composition of the disclosure induces a protective immunogenic response in the animal or a mammal (e.g., human) and stimulates both mucosal and systemic immune responses similar to those of the natural infection. Preferably that response includes the production of killer T-cell (T_{C} or CTL) responses, helper T-cell (T_{H}) responses, macrophages (MP), and specific antibody production in an inoculated subject.

Antigens of the disclosure may also be obtained or derived from the sequences of a pathogen such as, for example, multiple or combined epitopes of the proteins and/or polypeptides of coronaviruses, influenza viruses, or a combination thereof.

Antigens as disclosed herein include antigens containing “composite” epitopes, which are epitopes that are engineered. These epitopes are artificially created of two or more different epitopes, such that the resulting composite antigen has physical and/or chemical properties that differ from or are additive of the individual epitopes. These different epitopes may be of highly conserved regions of slightly different sequences of the same epitope of perhaps different serotypes. Oligonucleotides may also be constructed to express conserved regions of different epitopes (e.g., HA and NA of influenza virus, S and M of corona virus; or HA of influenza virus and S of coronavirus).

In some embodiments, antigens may be selected regions of influenza and/or SARS-COV-2 viruses that are known or believed to generate an effective immune response after administration. The peptide sequence of the antigen may contain a plurality of immunologically responsive regions or epitopes of one or more viruses, which are artificially arranged. Epitopes may be identical to known immunological regions of viruses, or entirely new constructs that have not previously existed and therefore artificially constructed. In one particular embodiment, the antigen of the disclosure induces a protective immunogenic response in the animal, mammal, or a subject (e.g., human) and stimulates both mucosal and systemic immune responses similar to those of the natural infection. In some embodiments, that response includes the production of killer T-cell (T_{C} or CTL) responses, helper T-cell (T_{H}) responses, macrophages (MP), and specific antibody production in an inoculated subject.

Antigens of the disclosure may be obtained or derived from the sequences of an influenza and/or SARS-COV-2 virus such as, for example, multiple or combined epitopes of the proteins and/or polypeptides. Antigens may be constructed of conserved regions of different epitopes (e.g., HA and NA of influenza virus; S and M of corona virus; or HA of influenza virus and S of coronavirus, etc.).

Antigens produced by expression of two or more oligonucleotides of the disclosure contain epitopes that represent two or more epitopes with epitope sequences only similar to the epitope sequences from which they were derived. Epitopes are regions obtained or derived from a protein or peptide of a virus that elicit a robust immunological response when administered to a subject. As used herein, the term “subject” refers to mammal or an animal including, but not limited to, Homo sapiens, simian, porcine, bovine, feline, canine, equine, etc. Often the subject is human. In some embodiments, robust response provides the subject with an immunological protection against developing disease from exposure to influenza virus and/or SARS-COV-2 virus. In some embodiments, compositions of the disclosure include an oligonucleotide that encodes an immune response stimulating antigen, wherein said antigen comprises the conserved regions or two related epitopes (e.g., HA and HA; NA and NA; M and M; S-protein and S-protein; etc.), or the conserved regions of two different epitopes (e.g., HA and NA; HA and M; NA and M; HA, NA, and S-protein; HA and S-protein; NA and S-protein; etc.). Antigens may also contain the variable regions which differ.

Antigens may include one or more T-cell stimulating epitopes, such as diphtheria toxoid, tetanus toxoid, a polysaccharide, a lipoprotein, or a derivative or any combination thereof (including fragments or variants thereof). Typically, the at least one sequence of the antigen is contained within the same molecule as the T-cell stimulating epitopes. In the case of protein-based T-cell stimulating epitopes, at least one repeated sequence of the composite antigen may be contained within the same polypeptide as the T-cell stimulating epitopes, may be conjugated thereto, or may be associated in other ways. In some embodiments, one or more T-cell stimulating epitopes are positioned at either the N-Terminus or the C-Terminus (or both) of the antigen.

In some embodiments, antigens may be peptide variants of naturally occurring sequences. As used herein, the term “variant” refers to an antigen that contains one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group; (3) replacement of one or more Val, Ile, Leu, Met, Ala, or Phe residues with one or more residues from the same group; (4) replacement of one or more Lys, Arg, or His residues with one or more residues from the same group; and (5) replacement of one or more Phe, Tyr, Trp, or His residues with one or more residues from the same group. A variant may also, or alternatively, contain non-conservative changes, for example, by substituting one of the amino acid residues from group (1) with an amino acid residue from group (2), group (3), group (4), or group (5). Variants may also (or alternatively) be modified by, for example, the deletion or addition of amino acids that have minimal influence on the immunogenicity, secondary structure and hydropathic nature of the peptide.

Another aspect of the disclosure provides polynucleotides including DNA, RNA (e.g., cRNA, mRNA), and PNA (peptide nucleic acid) constructs that encode the antigens of the disclosure. These polynucleotides may be single-stranded (coding or antisense) or double-stranded, and may be DNA (genomic, cDNA or synthetic) or RNA molecules. Additional coding or non-coding sequences may, but need not, be present within a polynucleotide of the present disclosure, and a polynucleotide may, but need not, be linked to other molecules and/or support materials. As is appreciated by those of ordinary skill in the art that, as a result of the degeneracy of the genetic code, there are many nucleotide sequences that encode a given primary amino acid sequence. Some of these polynucleotides bear minimal homology to the nucleotide sequence of any native gene. Nonetheless, polynucleotides that vary due to differences in codon usage are specifically contemplated in the present disclosure. Polynucleotides that encode an immunogenic peptide may generally be used for production of the peptide, in vitro or in vivo. Any polynucleotide may be further modified to increase stability in vivo. Possible modifications include, but are not limited to, the addition of flanking sequences at the 5′- and/or 3′-ends; the use of phosphorothioate or 2′-o-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Vaccine compositions of the disclosure contain the genetic sequence of two or more antigens as cRNA or mRNA, or DNA, plus other necessary sequences that provide for the expression of the antigens in cells. By injecting the mammal with the genetically engineered nucleic acid, antigens are produced in or on cells, which the subject's immune system recognizes and thereby generates a humoral or cellular response to antigens, and therefore the virus. Nucleic acid vaccines have a number of advantages over conventional vaccines, including the ability to induce a more general and complete immune response in the subject.

In some embodiments, the vaccine compositions of the disclosure comprise a pharmaceutically acceptable excipient, such as an adjuvant. An adjuvant refers to a vehicle used to enhance antigenicity. In some embodiments, an adjuvant can include a suspension of minerals (alum, aluminum hydroxide, or phosphate) on which antigen is adsorbed; or water-in-oil emulsion, for example, in which vaccine solution is emulsified in mineral oil. In some embodiments, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22 (9): 1004-1012, 2015). Additional adjuvants for use in the disclosed immunogenic compositions include the QS21 purified plant extract, Matrix M, MF59, ALFQ, ALFA, AS01, AS10b, and/or combinations, derivatives, and modifications thereof. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-a, IFN-y, G-CSF, L FA-3, CD72, B7-1, B7-2, OX-40L, 4-1 BBL and toll-like receptor (TLR) agonists, such as TLR-9 agonists. Additional description of adjuvants can be found, for example, in Singh (ed.) Vaccine Adjuvants and Delivery Systems. Wiley-Interscience, 2007). Adjuvants can be used in combination with the disclosed compositions. The formulation of pharmaceutically-acceptable excipients and carrier solutions is well known to those of ordinary skill in the art, as is the development of suitable dosing and treatment regimens for using the particular compositions described herein in a variety of treatment regimens. In one particular embodiment, vaccine compositions of the disclosure include ALFQ and may be given by IM, SQ, Intradermal or intranasal administration or in a manner compatible with the dosage formulation, and in such an amount as will be prophylactically or therapeutically effective and preferably immunogenic. The quantity to be administered depends on the subject to be treated, including, e.g., the capacity of the immune system to mount an immune response, and the degree of protection desired. Suitable dosage ranges may be on the order of several hundred micrograms (μg) of active ingredient per subject. Exemplary amounts of administration can range from about 0.1 μg to 2000 μg (even though higher amounts, such as, e.g., in the range of about 1 to about 10 mg are also contemplated), from about 0.5 μg to 1000 μg, from about 1 μg to about 500 μg, or from about 10 μg to about 100 μg. Suitable regimens for initial administration and booster shots are also variable but are typified by an initial administration followed by optional but preferred subsequent inoculations or other periodic administrations.

The amount of vaccine composition(s) and the time needed for the administration of such vaccine composition(s) will be within the purview of the ordinary-skilled artisan having benefit of the present teachings. The administration of a therapeutically-effective, pharmaceutically-effective, and/or prophylactically-effective amount of the disclosed vaccine compositions may be achieved by a single administration. Alternatively, in some circumstances, it may be desirable to provide multiple, or successive administrations of the immunogenic compositions, either over a relatively short, or even a relatively prolonged period of time, as may be determined by the skilled person overseeing the administration of such compositions.

An effective dose comprises amounts in the range of about 1 μg to about 1 mg per subject. In one exemplary embodiment, the vaccine dosage range is about 0.1 μg to about 10 mg per subject. However, one may prefer to adjust dosage based on the amount of oligonucleotides delivered. In either case, these ranges are merely guidelines from which one of ordinary skill in the art may deviate according to conventional dosing techniques. Precise dosages may be determined by assessing the immunogenicity of the conjugate produced in the appropriate host so that an immunologically effective dose is delivered. An immunologically effective dose is one that stimulates the immune system of the subject to establish an immune response to the immunogenic composition or vaccine. In some embodiments, a level of immunological memory sufficient to provide long-term protection against disease caused by influenza and/or SARS-COV-2 virus infection is obtained. Vaccines of the disclosure may be formulated with an adjuvant. By “long-term” it is preferably meant over a period of time of at least about 6 months, over at least about 1 year, over at least about 2 to 5 or even at least about 2 to about 10 years or longer.

In one particular aspect of the disclosure, a recombinant adenovirus (rAd) is provided. The rAd of the disclosure is adapted for use in preventing infection or transmission, or reducing severity of disease caused by influenza virus and/or SARS-Cov-2 virus in a subject. In some embodiments, the recombinant adenovirus comprises an oligonucleotide that encodes an immune response stimulating antigen in the subject. In particular, the recombinant adenovirus comprises at least two different oligonucleotides each of which independently comprises:
(i) a first oligonucleotide that encodes a hemagglutinin of influenza A virus or an immunogenic portion, variant, mutant, or fragment thereof;
(ii) a second oligonucleotide that encodes a neuraminidase of influenza A virus or an immunogenic portion, variant, mutant, or fragment thereof;
(iii) a third oligonucleotide that encodes a hemagglutinin of a first influenza B virus or an immunogenic portion, variant, mutant, or fragment thereof;
(iv) a fourth oligonucleotide that encodes a hemagglutinin of a second influenza B virus or an immunogenic portion, variant, mutant, or fragment thereof;
(v) a fifth oligonucleotide that encodes a spike protein (“S-protein”) or an immunogenic portion, variant, mutant, or fragment thereof of a first SARS-COV-2 virus; and
(vi) a sixth oligonucleotide that encodes an S-protein or an immunogenic portion, variant, mutant, or fragment thereof of a second SARS-COV-2 virus.

In general, the necessary oligonucleotide sequences for influenza and/or SARS-COV-2 viruses (e.g., the first-sixth oligonucleotides) can be obtained from the World Health Organization (WHO) and/or the Center for Disease Control (CDC). Accordingly, compositions of the disclosure can be modified as needed to provide vaccines for any future dominant or emerging strains of influenza and/or SARS-COV-2 viruses.

In some embodiments, at least one of the oligonucleotides encodes a SARS-COV-2 S protein, such as a stabilized form of the SARS-COV-2 S protein. In some embodiments, at least one of the oligonucleotides encodes at least a portion or immunogenic fragment of a SARS-COV-2 virus spike protein (S protein) or an immunogenic fragment or variant thereof having a sequence at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SARS-COV-2 virus spike protein portion (or immunogenic portion or variant thereof) of SEQ ID NO: 2; or at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 2 having K986P and V987P mutations; or SEQ ID NO: 2 having a D614G mutation. In some embodiments, at least one of the oligonucleotides encodes a spike protein of a SARS-COV-2 variant, in non-limiting examples a spike protein having a D80G, 144del, F157S, L5F, T95I, A67V, S477N, 144del, Q677H, A701V, F888L, T791 I, T859N, D950H, E484Q, D614G, E484K, N501Y, D69-70, L452R, or K417N or RBD E484K mutation relative to SEQ ID NO: 2. In some embodiments, at least one of the oligonucleotides encodes a spike protein from a WA1/2020, B.1.1.7, B.1.351, B.1.1.28, P.1, B.1.427, B.1.526, B.1.526.1, B.1.525, P.2, B.1.617, B.1.617.1, B.1.617.2, B.1.617.3, B.1.429, or B.1.429 variants. In other embodiments, the spike protein is further modified to be a prefusion stabilized form (e.g., having double proline substitution between residues 1050 to 1069 or between residues 981 to 999).

The vector disclosed herein also includes conventional control elements necessary which are operably linked to two or more oligonucleotides that encode immune response stimulating antigen in a manner that permits its transcription, translation and/or expression in a cell transfected with the plasmid vector or infected with the virus produced by the present disclosure. As will be recognized, the term “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product.

A great number of expression control sequences, including promoters which are native, constitutive, inducible and/or tissue-specific, are known in the art and may be utilized. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the b-actin promoter, the phosphoglycerol kinase (PGK) promoter, and the EF1a promoter [Invitrogen]

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. For example, inducible promoters include the zinc-inducible sheep metallothionine (MT) promoter and the dexamethasone (Dex)-inducible mouse mammary tumor virus (MMTV) promoter. Other inducible systems include the T7 polymerase promoter system [WO 98/10088]; the ecdysone insect promoter [No et al, Proc. Natl. Acad. ScL USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Flarvey et al, Curr. Opin. Chem. Biol., 2:512-518 (1998)]. Other systems include the FK506 dimer, VP 16 or p65 using castradiol, diphenol murislerone, the RU486-inducible system [Wang et al, Nat. Biotech., 15:239-243 (1997) and Wang et al, Gene Ther, 4:432-441 (1997)] and the rapamycin-inducible system [Magari et al, J. Clin. Invest., 100:2865-2872 (1997)]. The effectiveness of some inducible promoters increases over time. In such cases one can enhance the effectiveness of such systems by inserting multiple repressors in tandem, e.g., TetR linked to a TetR by an IRES. Alternatively, one can wait at least 3 days before screening for the desired function. One can enhance expression of desired proteins by known means to enhance the effectiveness of this system. For example, using the Woodchuck Hepatitis Virus Posttranscriptional Regulatory Element (WPRE).

In other embodiments, the native promoter for the oligonucleotide will be used. The native promoter may be preferred when it is desired that expression of the oligonucleotide (i.e., transgene) should mimic the native expression. The native promoter may be used when expression of the transgene must be regulated temporally or developmental, or in a tissue-specific manner, or in response to specific transcriptional stimuli. In a further embodiment, other native expression control elements, such as enhancer elements, polyadenylation sites or Kozak consensus sequences may also be used to mimic the native expression. Another embodiment of the transgene includes a transgene operably linked to a tissue-specific promoter. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally occurring promoters. Other components of the vector may include an origin of replication. Selection of these and other promoters and vector elements are conventional and many such sequences are available and are well known to one of ordinary skill in the art [see, e.g., Sambrook et al, and references cited therein]. These vectors are generated using the techniques and sequences provided herein, in conjunction with techniques known to those of skill in the art. Such techniques include conventional cloning techniques of cDNA such as those described in texts [Sambrook et al, Molecular Cloning: A Laboratory Manual, Cold Spring Harbor Press, Cold Spring Harbor, NY], use of overlapping oligonucleotide sequences of the adenovirus genomes, polymerase chain reaction, and any suitable method which provides the desired nucleotide sequence.

In some embodiments, the adenoviral vector of the disclosure comprises at least one oligonucleotides encoding an immune response stimulating antigen that is expressed by the nucleic acid sequence of SEQ ID NO: 1; or an oligonucleotide encoding an immune response stimulating antigen that is expressed by the nucleic acid sequence having at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 1. Still in other embodiments, the rAd vector of the disclosure comprises at least one oligonucleotide encoding an immune response stimulating antigen that is expressed by the nucleic acid sequence has at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SARS-COV-2 virus spike protein portion of WA1/2020, B.1.1.7, B.1.351, B.1.1.28, P.1, B.1.427, B.1.526, B.1.526.1, B.1.525, P.2, B.1.617, B.1.617.1, B.1.617.2, B.1.617.3, B.1.429, or B.1.429 variant.

Still in other embodiments, the immune response stimulating antigen that is encoded by at least one of the oligonucleotides in rAd vector has protein sequence of SEQ ID NO: 2. Yet in other embodiments, the immune response stimulating antigen that is encoded by at least one of the oligonucleotides in rAd vector has protein sequence that has at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 2. Still in other embodiments, the immune response stimulating antigen that is encoded by at least one of the oligonucleotides in rAd vector has protein sequence 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SARS-COV-2 virus spike protein WA1/2020, B.1.1.7, B.1.351, B.1.1.28, P.1, B.1.427, B.1.526, B.1.526.1, B.1.525, P.2, B.1.617, B.1.617.1, B.1.617.2, B.1.617.3, B.1.429, or B.1.429 variant.

In some embodiments, the recombinant adenovirus comprises (i) at least one oligonucleotide that encodes a hemagglutinin or an immunogenic portion, variant, mutant, or fragment thereof of influenza A or B and (ii) an S-protein or an immunogenic portion, variant, mutant, or fragment thereof of a SARS-COV-2 virus. Still in other embodiments, the recombinant adenovirus comprises (i) at least two oligonucleotide that encodes a hemagglutinin or an immunogenic portion, variant, mutant, or fragment thereof of influenza A or B and (ii) two different S-protein or an immunogenic portion, variant, mutant, or fragment thereof of SARS-CoV-2 viruses.

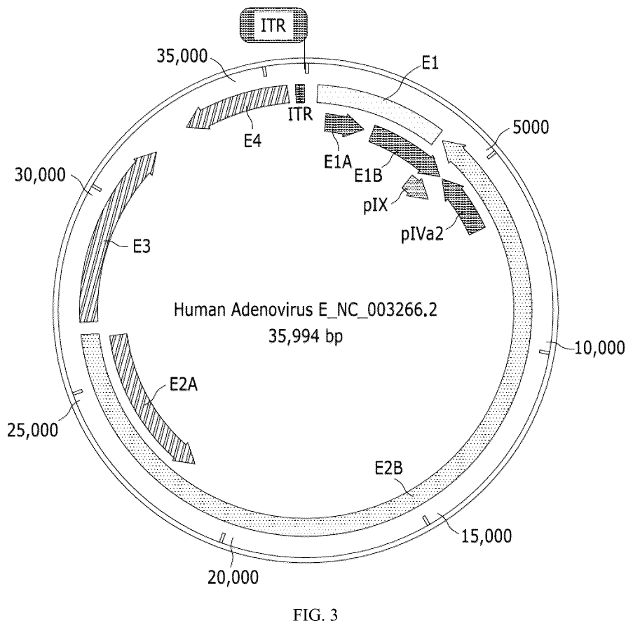

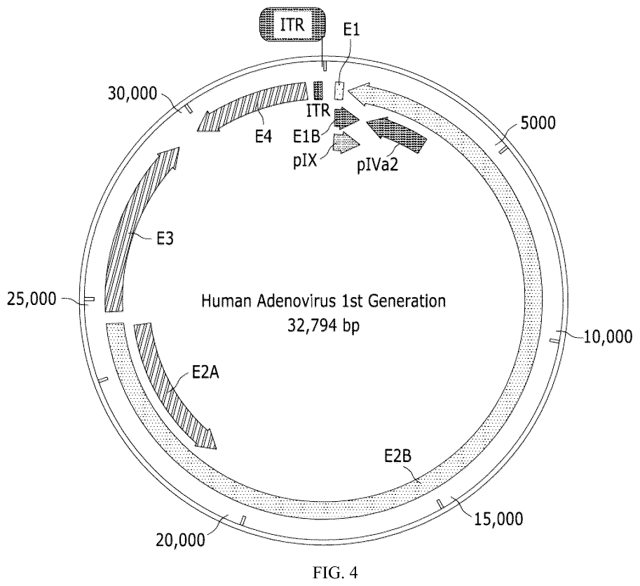

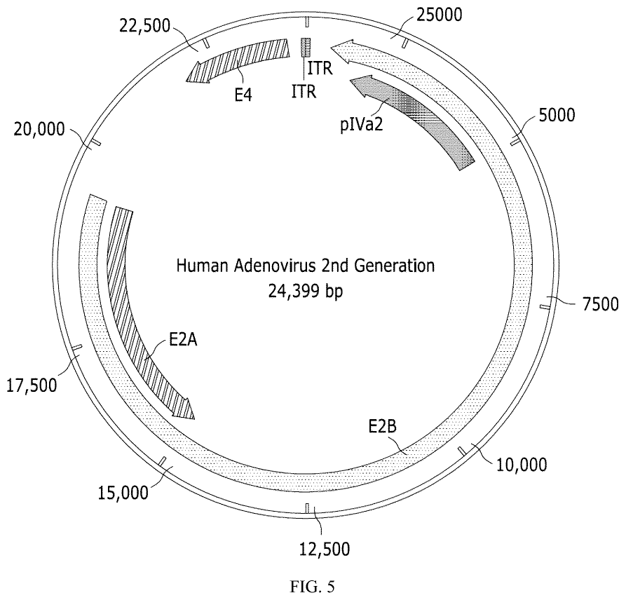

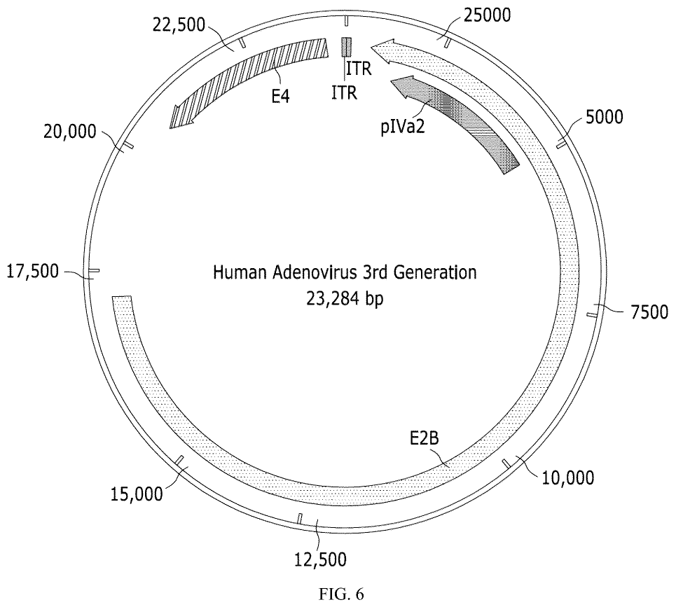

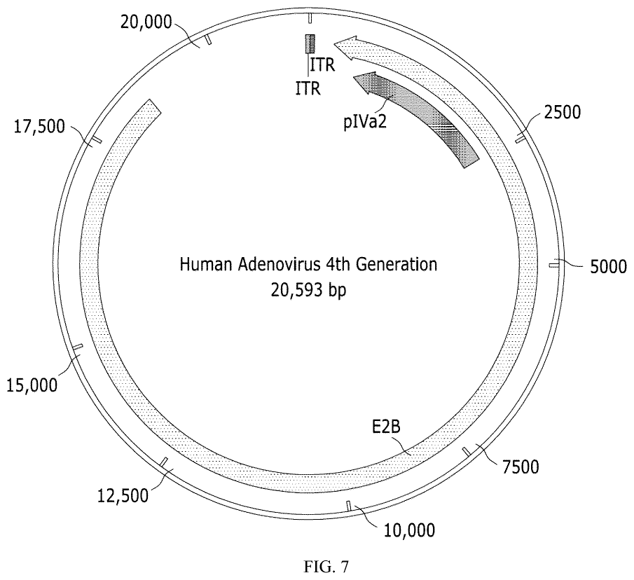

Yet in some embodiments, the adenovirus is selected from the group consisting of: (i) a first, second, third, or fourth-generation human adenovirus 5 (see FIGS. 3-7, respectively); (ii) a rare-serotype adenovirus; (iii) a nonhuman adenovirus; and (iv) a combination thereof.

In some embodiments, the recombinant adenovirus is adapted for transfecting a host cell via intramuscular, intranasal, or inhalation route.

In one particular embodiment, the first oligonucleotide encodes at least one of 18 hemagglutinin (HA) subtypes 1-18 or an immunogenic portion, variant, mutant, or fragment thereof. In another embodiments, the second oligonucleotide encodes at least one of 10 neuraminidase (NA) subtypes 1-10 or an immunogenic portion, variant, mutant, or fragment thereof. Sill in another embodiment, the third oligonucleotide encodes B/Yamagata/16/88 HA or an immunogenic portion, variant, mutant, or fragment thereof. Yet in another embodiment, the fourth oligonucleotide encodes B/Victoria/2/87 HA or an immunogenic portion, variant, mutant, or fragment thereof. In further embodiment, the fifth oligonucleotide encodes said S-protein of one of SARS-COV-2 variants B.1.1.7, B.1.351, B1.1.28-P.1, B.1.617.2, B.1.1.529, BA.4, BA.5, BQ1.1, XBB.1.5, BA.2.75.2, or an immunogenic portion, variant, mutant, or fragment thereof. Yet still in another embodiment, the sixth oligonucleotide encodes said S-protein of SARS-COV-2 variant that is different from said fifth oligonucleotide.

Still in other embodiments, the recombinant adenovirus comprises at least three different oligonucleotides that encode three different immune response stimulating antigens or is trivalent (i.e., tricistronic). In other embodiments, the recombinant adenovirus comprises at least four different oligonucleotides or is quadrivalent (i.e., quadricistronic). Yet in other embodiments, the recombinant adenovirus comprises at least five different oligonucleotides or is pentavalent (i.e., pentacistronic). In further embodiments, the recombinant adenovirus comprises at least six different oligonucleotides or is hexavalent (i.e., hexacistronic).

Exemplary adenoviruses that are useful in the disclosure include, but are not limited to, a first, second, third, or fourth-generation human adenovirus 5 (FIGS. 3-7, respectively); a rare-serotype adenovirus; a nonhuman adenovirus; and a combination thereof. Exemplary rare-serotype adenoviruses that are useful in the disclosure include, but not limited to, Ad11, Ad26, Ad35, Ad48, Ad49, Ad50, and a combination thereof. Exemplary nonhuman adenoviruses that are useful in the disclosure include, but are not limited to, simian Ad36, bovine Ad3, canine Ad2, porcine Ad3, and a combination thereof. In one particular embodiment, the nonhuman adenovirus comprises simian Ad36.

Yet in other embodiments, the recombinant adenovirus further comprises 5′- and 3′-inverted terminal repeats (ITR) and a packaging signal (v).

Still in other embodiments, the recombinant adenovirus further includes (i) a promoter, (ii) an enhancer, (iii) a polyadenylation moiety, (iv) an internal ribosome entry site (IRES), (v) a self-cleaving protein site, or (vi) a combination thereof. In some embodiments, the self-cleaving protein site comprises T2A, P2A, E2A, F2A, or a combination thereof. Yet in other embodiments, the polyadenylation moiety comprises simian virus 40 (SV40) polyadenylation (PolyA) moiety, bovine growth hormone (bGH) PolyA moiety, or a combination thereof.

In further embodiments, the recombinant adenovirus further comprises a cytomegalovirus (CMV) promoter or enhancer, an elongation factor 1a (EF1a), a chicken β-actin (CBA) promoter, a CAG promotor, or a combination thereof.

Another aspect of the disclosure provides a plasmid comprising an adenovirus genome that is modified to comprise a transgene that is operatively linked to expression control sequences which direct transcription, translation, and/or expression in a host cell. Typically, the transgene comprises at least two different oligonucleotides that encode immune response stimulating antigens (e.g., antigenic proteins). Each of the oligonucleotide that encode an immune response stimulating antigen is different from one another and is independently selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is a different strain than said first influenza B virus;
(v) an oligonucleotide that encodes a spike protein (S-protein) or an immunogenic portion, variant, mutant, or fragment thereof of a first SARS-COV-2 virus; and
(vi) an oligonucleotide that encodes an S-protein or an immunogenic portion, variant, mutant, or fragment thereof of a second SARS-COV-2 virus, wherein said second SARS-COV-2 virus is a different strain than said first SARS-COV-2 virus.

In some embodiments, the genome is derived from an adenovirus selected from the group consisting of: (i) a first, second, third, or fourth-generation human adenovirus 5; (ii) a rare-serotype adenovirus; (iii) a nonhuman adenovirus; and (iv) a combination thereof. In one particular embodiment, the adenovirus is a nonhuman adenovirus.

Exemplary nonhuman adenoviruses that are used include, but not limited to, simian Ad36, bovine Ad3, canine Ad2, porcine Ad3, or a combination thereof. In one specific embodiment, the nonhuman adenovirus comprises simian Ad36. In another embodiment, the genome of simian Ad36 lacks a native E1 and optionally, the E3 or E3B locus.

Still in other embodiments, at least one of the oligonucleotides encodes at least one of 18 hemagglutinin (HA) subtypes 1-18 or an immunogenic portion, variant, mutant, or fragment thereof. Yet in other embodiments, at least one of the oligonucleotides encodes at least one of 10 neuraminidase (NA) subtypes 1-10 or an immunogenic portion, variant, mutant, or fragment thereof. In other embodiments, at least one of the oligonucleotides encodes B/Yamagata/16/88 HA or an immunogenic portion, variant, mutant, or fragment thereof. In another embodiment, at least one of the oligonucleotides encodes B/Victoria/2/87 HA or an immunogenic portion, variant, mutant, or fragment thereof. Yet in further embodiments, at least one of the oligonucleotides encodes said S-protein of one of SARS-COV-2 variants B.1.1.7, B.1.351, B1.1.28-P.1, B.1.617.2, B.1.1.529, BA.4, BA.5, BQ1.1, XBB.1.5, BA.2.75.2, or an immunogenic portion, variant, mutant, or fragment thereof. Still yet in other embodiments, at least one of the oligonucleotides encodes said S-protein of a different SARS-COV-2 variant.

The plasmid can comprise at least three, four, five, or six different antigenic encoding oligonucleotides.

Yet another aspect of the disclosure provides a recombinant adenovirus (rAd) vector comprising a genome of an adenovirus, wherein said genome of the adenovirus has been modified such that said genome comprises at least two different extraneous oligonucleotides that is operatively linked to expression control sequences which direct transcription, translation, and/or expression in a host cell, and wherein each of said extraneous oligonucleotide is different from other extraneous oligonucleotides and is independently selected one of the following oligonucleotides:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is different strain compared to said first influenza B virus;
(v) an oligonucleotide that encodes a spike protein (S-protein) or an immunogenic portion, variant, mutant, or fragment thereof of a first SARS-COV-2 virus; and
(vi) an oligonucleotide that encodes an S-protein or an immunogenic portion, variant, mutant, or fragment thereof of a second SARS-COV-2 virus, wherein said second SARS-COV-2 virus is a different strain than said first SARS-COV-2 virus.

In some embodiments, the adenovirus is selected from the group consisting of (i) a first, second, third, or fourth-generation human adenovirus 5; (ii) a rare-serotype adenovirus; (iii) a nonhuman adenovirus; and (iv) a combination thereof.

Still in other embodiments, the rAd vector further comprises naturally occurring adenovirus major serotype capsid proteins (i.e., hexon, penton base, and fiber) and four minor proteins (i.e., IIIa, VI, VIII, and IX).

In some embodiments, the rAd vector is adapted for transfecting a host cell via intramuscular, intranasal, or inhalation route.

Yet in other embodiments, nonhuman adenovirus comprises simian Ad36, bovine Ad3, canine Ad2, porcine Ad3, or a combination thereof. In one particular embodiment, the nonhuman adenovirus comprises simian Ad36.

In further embodiments, the rAd vector further comprises 5′ and 3 inverted terminal repeats (ITR) and a packaging signal (v).

Still in other embodiments, the rAd vector further comprises (i) a promoter, (ii) an enhancer, (iii) a polyadenylation moiety, (iv) an internal ribosome entry site (IRES), (v) a self-cleaving protein site, or (vi) a combination thereof. In one particular embodiment, the self-cleaving protein site comprises T2A, P2A, E2A, F2A, or a combination thereof. In another embodiments, the polyadenylation moiety comprises simian virus 40 (SV40) polyadenylation (PolyA) moiety, bovine growth hormone (bGH) PolyA moiety, or a combination thereof.

Yet in other embodiments, the rAd vector further comprises a cytomegalovirus (CMV) promoter or enhancer, an elongation factor 1a (EF1a), a chicken β-actin (CBA) promoter, a CAG promotor, or a combination thereof.

In other embodiments, at least one of the oligonucleotides of the rAd vector encodes at least one of 18 hemagglutinin (HA) subtypes 1-18 or an immunogenic portion, variant, mutant, or fragment thereof. Still in other embodiments, at least one of the oligonucleotides of the rAd vector encodes at least one of 10 neuraminidase (NA) subtypes 1-10 or an immunogenic portion, variant, mutant, or fragment thereof. Yet in other embodiments, at least one of the oligonucleotides of the rAd vector encodes B/Yamagata/16/88 HA or an immunogenic portion, variant, mutant, or fragment thereof. In further embodiments, at least one of the oligonucleotides of the rAd vector encodes B/Victoria/2/87 HA or an immunogenic portion, variant, mutant, or fragment thereof. Still in other embodiments, at least one of the oligonucleotides of the rAd vector encodes said S-protein of a first SARS-COV-2 strain variants B.1.1.7, B.1.351, B1.1.28-P.1, B.1.617.2, B.1.1.529, BA.4, BA.5, BQ1.1, XBB.1.5, BA.2.75.2, or an immunogenic portion, variant, mutant, or fragment thereof. In yet other embodiments, at least one of the oligonucleotides of the rAd vector encodes said S-protein of a different strain of SARS-COV-2 virus.

In further embodiments, the recombinant adenovirus comprises at least three, at least four, at least five, or at least six different oligonucleotides that encode immune stimulating antigens.

In one particular embodiment, the rare-serotype adenovirus comprises Ad11, Ad26, Ad35, Ad48, Ad49, Ad50, or a combination thereof.

In further aspects of the disclosure provide a pharmaceutical composition comprising any one of a recombinant adenovirus (rAd) vector disclosed herein.

Yet other aspects of the disclosure provide a method for administering a flu or SARS-COV-2 virus vaccine, wherein said vaccine is a polyvalent vaccine comprising at least two different extraneous oligonucleotides, said method comprising administering a recombinant adenovirus (rAd) vector to a subject in need of such a vaccine, wherein said rAd vector comprises a genome of an adenovirus, and wherein said genome of the adenovirus has been modified such that said genome comprises at least two different extraneous oligonucleotides that is operatively linked to expression control sequences which direct transcription, translation, and/or expression in a host cell, and wherein each of said extraneous oligonucleotide encodes an immune stimulating antigen that is different from immune stimulating antigen(s) encoded by other extraneous oligonucleotide(s). Each extraneous oligonucleotide is independently selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is a different strain compared to said first influenza B virus;
(v) an oligonucleotide that encodes a spike(S) protein or an immunogenic portion, variant, mutant, or fragment thereof of a first SARS-COV-2 virus; and
(vi) an oligonucleotide that encodes an S protein or an immunogenic portion, variant, mutant, or fragment thereof of a second SARS-COV-2 virus, wherein said second SARS-COV-2 virus is a different strain compared to said first SARS-COV-2 virus.

In some embodiments, the vaccine is administered to the subject via intramuscular, intranasal, or inhalation route. Still in other embodiments, the vaccine is a bivalent or bicistronic vaccine. Yet in other embodiments, the vaccine is a trivalent or tricistronic vaccine. In further embodiments, the vaccine is a quadrivalent or quadricistronic vaccine. In other embodiments, the vaccine is a pentavalent or pentacistronic vaccine. Still yet in other embodiments, the vaccine is a hexavalent or hexacistronic vaccine.

Still in other embodiments, the method uses an adenovirus that is selected from the group consisting of: (i) a first, second, third, or fourth-generation human adenovirus 5; (ii) a rare-serotype adenovirus; (iii) a nonhuman adenovirus; and (iv) a combination thereof. In some embodiments, the method uses nonhuman adenovirus that comprises simian Ad36, bovine Ad3, canine Ad2, porcine Ad3, or a combination thereof. In one particular embodiment, the method uses simian Ad36.

Some of the specific oligonucleotides that can be used in compositions of the disclosure include any prevalent variant sequences of Influenza HA, NA proteins obtained from global surveillance agencies such as WHO and CDC. In general, there are four types of influenza viruses: A, B, C, and D. Typically, influenza A and B viruses cause seasonal (flu season) epidemics in the United States every winter. Of the four influenza viruses, only influenza A viruses cause flu pandemics that occur when a different influenza A virus spreads efficiently among people that have little or no immunity. Generally, influenza B viruses change their genetic and antigenic properties more slowly than influenza A viruses. Influenza C virus generally causes mild illness and is not thought to cause human epidemics. Influenza D virus primarily infects cattle and is not known to infect people.

In one particular embodiment, compositions of the disclosure are quadrivalent influenza vaccines that use antigens to target 4 influenza strains that cause a majority of the flu cases worldwide: two type A (H1N1 and H3N2) and two type B (Victoria and Yamagata lineages).

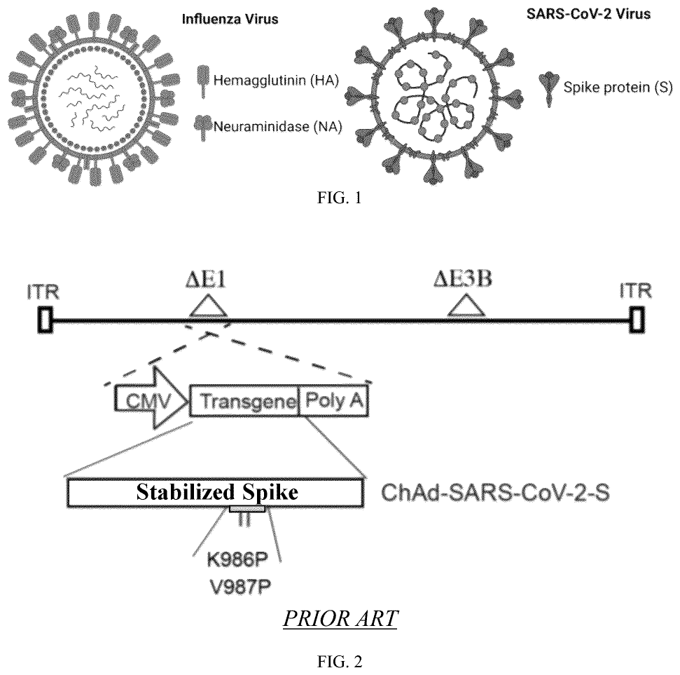

Influenza Viruses: Influenza A viruses are divided into subtypes based on two proteins on the surface of the virus: hemagglutinin (HA) and neuraminidase (NA). See, for example, FIG. 1. There are 18 HA subtypes (H1-H18) and 10 NA subtypes (N1-N10), with more than 130 influenza A subtype combinations identified in nature. Currently, A (H1N1) and A (H3N2) are the two subtypes of the influenza A virus that routinely circulate in people (seasonal flu). Based on the similarity of their HA gene sequences influenza A subtypes can be further broken down into different genetic clades and sub-clades helping flu experts track the proportion of viruses from different clades in circulation. Clades and sub-clades emerge as a result of high mutation rates in the type A influenza viruses which can lead to antigenically different viruses. This means that the host's immune response generated by infection or vaccination against one type A virus will not neutralize an antigenically different virus and will not protect against the other virus. Therefore, testing sites worldwide collect influenza samples, monitor antigenic drifts in HA and NA in real-time, and recommend current vaccine strains. Influenza B viruses are classified into two lineages instead of subtypes: B/Yamagata and B/Victoria, which can then be further classified into specific clades and sub-clades. Influenza B viruses generally change more slowly in terms of their genetic and antigenic properties than influenza A viruses. Recent surveillance data shows the co-circulation of both influenza B viruses in the United States and around the world. However, the proportion of influenza B viruses from each lineage varies by geographic location and by season.

The four major flu candidate vaccine viruses (CVVs) in use currently (2022-23) as recommended by the World Health Organization (WHO) are:
(i) A/Wisconsin/588/2019 (H1N1)
(ii) A/Darwin/6/2021 (H3N2)
(iii) B/Austria/1359417/2021 (B/Victoria lineage)
(iv) B/Phuket/3073/2013 (B/Yamagata lineage).
   Lists of prototype viruses for egg-propagated, cell culture-propagated, and recombinant-based vaccines together with candidate vaccine viruses (CVVs) suitable for use in human vaccine production are available on the WHO website and their sequences are available with the Global Initiative on Sharing Avian Influenza Data (GISAID).

SARS-COV-2 Virus: The SARS-COV-2 RNA genome is approximately 30,000 nucleotides in length. Two-thirds of the genes encode nonstructural proteins that enable genome replication and viral RNA synthesis. The remaining one-third encode structural proteins such as spike(S) (FIG. 1), envelope, membrane, and nucleoprotein (NP) that form the spherical virion, and accessory proteins that regulate cellular responses. The S-protein forms homotrimeric spikes on the virion and engages the cell-surface receptor angiotensin-converting enzyme 2 (ACE2) to promote coronavirus entry into human cells. The SARS-COV and SARS-COV-2 S proteins are cleaved sequentially during the entry process to yield S1 and S2 fragments, followed by further processing of S2 to yield a smaller S2′ protein. The S1 protein includes the receptor binding domain (RBD) and the S2 protein promotes membrane fusion. The structure of a soluble, stabilized prefusion form of the SARS-COV-2 S protein was solved by cryo-electron microscopy, revealing considerable similarity to the SARS-CoV S protein. This form of the S protein is recognized by potently neutralizing monoclonal antibodies and can serve as a vaccine target.

Severe acute respiratory syndrome coronavirus 2 (SARS-COV-2) is a positive-sense, single-stranded RNA virus that was first isolated in late 2019 from patients with severe respiratory illness in Wuhan, China. SARS-COV-2 is related to two other highly pathogenic respiratory viruses, SARS-COV and Middle East respiratory syndrome coronavirus (MERS-COV). SARS-COV-2 infection results in a clinical syndrome, coronavirus disease 2019 (COVID-19), that can progress to respiratory failure and may present with cardiac pathology, gastrointestinal disease, coagulopathy, or a hyperinflammatory syndrome. The elderly, immunocompromised, and those with certain co-morbidities (e.g., obesity, diabetes, and hypertension) are at greatest risk of death from COVID-19. SARS-COV-2 has created a global emergency due to the rapid worldwide spread of coronavirus disease 2019 (COVID-19). As of Jan. 6, 2023, SARS-COV-2 and its variants have caused more than 657 million infections and 6.68 million deaths, with the US alone accounting for 99.4 million cases and 1,082,265 deaths (covid19.who.int). In earlier SARS-COV-2 variants about 15% of infected patients developed pneumonia and around 5% of patients developed more critical symptoms such as acute respiratory distress syndrome and multiple organ failure. The Omicron variant (B.1.1.529) while being much more (2-5-fold) infective than the Delta (B.1.617.2) variant shows 2-5-fold less fatality, and caused significantly less morbidity, hospital admissions and requirement for oxygen supplementation in adults. However, the hospitalization rates for children were higher during the Omicron peak. Globally surveilled SARS-COV-2 sequences shared by the Global Initiative on Sharing All Influenza Data (GISAID), allow real-time tracking of SARS-COV-2 mutations and new variants. Omicron is currently the dominant variant circulating globally, accounting for >98% of viral sequences shared on GISAID after February 2022. Since its designation as a VOC by WHO on 26 Nov. 2021, viruses part of the Omicron complex have continued to evolve, leading to descendent lineages (BA.1, BA.1.1, BA.2, BA.3, BA.4 and BA.5) with different genetic constellations of mutations. As transmission of these VOCs has been sustained, this has led to significant intra-VOC evolution. Considering this, WHO has added a new category to its variant tracking system, termed “Omicron subvariants under monitoring” to alert public health authorities globally, about VOC lineages that may require prioritized attention and monitoring, such as BF.7, BF.14, BQ.1, BQ.1.1, BA.2.75, BA4.6, XBB and BA2.3.20. The main objective of this category is to investigate if these lineages may pose an additional threat to global public health as compared to other circulating viruses.

The rapid expansion and prolonged nature of the COVID-19 pandemic and its accompanying morbidity, mortality, and destabilizing socioeconomic effects have made the development and deployment of a SARS-COV-2 vaccine an urgent global health priority. Although multiple vaccines are available against the SARS-COV-2 including mRNA, Adenoviral and protein-based vaccine, the rate of virus evolution and transmission within the USA and globally remains high.

Most of the vaccines currently in use are administered via intramuscular route including Johnson & Johnson Ad26.COV2 and AstraZeneca ChAdOx1 nCOV-19 adenoviral platforms. Thus, questions remain as to the ability of these vaccines to curtail both transmission and severe disease, especially if upper airway infection is not prevented or reduced. While many of the intramuscular vaccines prevent SARS-COV-2-induced pneumonia in non-human primates, their effectiveness against upper airway infection and, presumably, transmission remains uncertain. For example, many of the IM-administered vaccines showed variable protection against upper-airway infection and transmission in pre-clinical studies and failed to induce substantive mucosal (immunoglobulin A [IgA]) immunity. This is important because more transmissible SARS-COV-2 variants have substitutions in the spike protein that lead to diminished neutralization by vaccine-induced sera. Besides the negative impacts on protection, the combination of diminished immunity against variants and naturally lower anti-S IgG levels in the respiratory mucosa causes further selection of vaccine resistance in the upper airway and increases transmission into the general population.

The spike(S) protein of the SARS-COV-2 virion engages the cell-surface receptor angiotensin-converting enzyme 2 (ACE2) to promote coronavirus entry into human cells. Because the S protein is critical for viral entry, it has been targeted for vaccine development and therapeutic antibody interventions. SARS-COV-2 S protein is cleaved to yield S1 and S2 fragments, followed by further processing of S2 to yield a smaller S20 protein. The S1 protein includes the receptor-binding domain (RBD), and the S2 protein promotes membrane Fusion. Spike proteins in vaccines are stabilized in the prefusion form by either the S-2P or hexapro mutations. The prefusion stabilized form of the SARS-COV-2 S protein, which displays the RBD in an “up” position, (exposing the RBD to immune surveillance) is recognized by potently neutralizing monoclonal antibodies or protein inhibitors, resulting in robust systemic and mucosal immunity. Unstabilized SARS-COV-2 S protein prematurely refolds to the post-fusion conformation, compromising immunogenic properties.

The nucleotide sequence of surface glycoprotein or the spike(S) protein of Wuhan-Hu-1 SARS-COV-2 virus along with the corresponding protein sequence are disclosed in SEQ ID NO:1 (accession code: YP_009724390.1) and SEQ ID NO:2, respectively. Following are some examples of SARS-COV-2 virus mutations, relative to Wuhan-Hu-1 SARS-CoV02 virus.

### Alpha Variant (B.1.1.7-UK Origin)

S1 domain: Deletion H69-V70, deletion Y144, A570D, D614G, P681H
RBD: N501Y
S2 domain: T716I, S982A, D1118H

### Beta Variant (B.1.351-South African Origin)

S1 domain: L18F, D80A, D215G, deletion L242-L244, D614G
RBD: K417N, E484K, N501Y
S2 domain: A701V

### Delta Variant (B.1.617-Indian Origin)

S1 domain: T19R, T95I, G142D, E156G, deletion (A) F157-R158, D614G, P681R
RBD: LA52R, T478K
S2 domain: D950N

### Omicron BA.1 Variant (B.1.529-BA.1)

S1 domain: A67V, deletion (A) H69-V70, T95I, G142D, ΔV143-Y145, ΔN211, L212I, ins214EPE, T547K, D614G, H655Y, N679K, P681H
RBD: G339D, S371L, S373P, S375F, K417N, N440K, G446S, S477N, T478K, E484A, Q493R, Q498R, N501Y, Y505H
S2 domain: N764K, D796Y, N856K, Q954H, N969K, L981F

### Omicron BA.4/BA.5 Variant (B.1.529-BA.4/BA.5)

S1 domain: T19I, deletion (Δ) L24-P26, A27S, ΔH69+V70, G142D, V213G, D614G, H655Y, N679K, P681H
RBD: G339D, S371F, S373P, S375F, T376A, D405N, R408S, K417N, N440K, L452R, S477N, T478K, E484A, F486V, Q498R, N501Y, Y505H
S2 domain: N764K, D796Y, Q954H, N969K

The vectors of present include appropriate sequences operably linked to the coding sequence or ORF to promote expression of two or more immune response stimulating antigens in a targeted host cell or a subject. As used herein, the term “subject” refers to a mammal, such as Homo sapiens, canine, feline, simian, equine, bovine, porcine, etc. Typically, the subject is human. “Operably linked” sequences include both expression control sequences such as promoters that are contiguous with the coding sequences and expression control sequences that act in trans or distally to control the expression of the polypeptide product.

Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., Kozak consensus sequence); sequences that enhance nucleic acid or protein stability; and when desired, sequences that enhance protein processing and/or secretion. Many varied expression control sequences, including native and non-native, constitutive, inducible and/or tissue-specific, are known in the art and may be utilized herein. depending upon the type of expression desired.

Expression control sequences for eukaryotic cells typically include a promoter, an enhancer, such as one derived from an immunoglobulin gene, SV40, CMV, etc., and a polyadenylation sequence which may include splice donor and acceptor sites. The polyadenylation sequence generally is inserted 3′- to the coding sequence and 5′- to the 3′-ITR sequence. PolyA from bovine growth hormone or others may be used.

The promoter may be selected from a number of constitutive or inducible promoters that can drive expression of the selected oligonucleotides.

The rAd used in the present disclosure may be constructed and produced using the materials and methods described herein and those well-known in the art. The methods that are typically employed for producing any construct of this disclosure are conventional and include genetic engineering, recombinant engineering, and synthetic techniques readily understood by the ordinarily skilled artisan.

The present disclosure also provides pharmaceutical compositions. The pharmaceutical composition comprises an adenoviral composition of the present disclosure, as an active ingredient, and at least one pharmaceutically acceptable excipient. The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, trehalose, dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non limiting examples of suitable lubricants include minerals such as talc or silica; and fats such as vegetable stearin, magnesium stearate, or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers. The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopcial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used. The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Many of the IM-administered vaccines for SARS-COV-2 virus showed variable protection against upper-airway infection and transmission in pre-clinical studies and failed to induce substantive mucosal (immunoglobulin A [IgA]) immunity. Accordingly, in some embodiments, compositions of the disclosure are delivered intranasally because more transmissible SARS-COV-2 variants have been shown to have substitutions in the spike protein that lead to diminished neutralization by vaccine-induced sera. Besides the negative impacts on protection, the combination of diminished immunity against variants and naturally lower anti-S IgG levels in the respiratory mucosa causes further selection of vaccine resistance in the upper airway and increases transmission into the general population. As used herein, the term “composition” when referring to those of the present disclosure refers to any composition, rAd, recombinant virus vector, plasmid, pharmaceutical composition, or any other materials disclosed herein that comprise two or more oligonucleotides disclosed herein.

A formulation comprising a composition for intranasal deliver may have a pH corresponding to a physiologically acidic nasal pH. The physiologically acidic nasal pH may depend on intact nasal mucosal function. A composition may comprise a pH of about be 6.5±0.5 (5.9 to 7.3) or about 6.7±0.6 (5.3 to 7.6). A composition may comprise a pH of about 3.8-7.7 (mean±SD 5.7±0.9). A composition for nasal deliver may be in the slightly acidic range. The average pH may have an acidity of pH 5.7.

Effective delivery of therapeutic agents via intranasal administration must take into account the decreased transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant disclosure optionally incorporate effective mucolytic or mucus-clearing agents, which serve to degrade, thin or clear mucus from intranasal mucosal surfaces to facilitate absorption and/or adsorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the disclosure, or as an additive within a combinatorial formulation of the disclosure, to provide an improved formulation that enhances intranasal delivery of biotherapeutic compounds by reducing the barrier effects of intranasal mucus.

A variety of mucolytic or mucus-clearing agents are available for incorporation within the methods and compositions of the disclosure. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the disclosure include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the disclosure include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein a-amylase and b-amylase are representative of this class of enzymes, although their mucolytic effect may be limited. In contrast, bacterial glycosidases which allow these microorganisms to permeate mucus layers of their hosts.

The composition can be formulated into various dosage forms and administered by a number of different means that will deliver a therapeutically effective amount of the active ingredient. Such compositions can be administered orally (e.g., inhalation), or parenterally in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

For parenteral administration (including subcutaneous, intraocular, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

Generally, a safe and effective amount of an adenoviral composition is administered, for example, that amount that would cause the desired therapeutic effect in a subject while minimizing undesired side effects. In various embodiments, an effective amount of an adenoviral composition described herein can substantially reduce viral infectivity in a subject suffering from a viral infection. In some embodiments, an effective amount is an amount capable of treating a respiratory viral infection. In some embodiments, an effective amount is an amount capable of treating one or more symptoms associated with a respiratory viral infection.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Dosages of the adenoviral vector will depend primarily on factors such as the condition being treated, the age, weight and health of the patient, and may thus vary among patients. For example, a therapeutically effective adult human or veterinary dosage of the viral vector is generally in the range of from about 100 pL to about 100 mL of a carrier containing concentrations of from about 1×10⁶ to about 1×10¹⁵ particles, about 1×10⁷ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending the route of administration, and the therapeutic or vaccinal application for which the recombinant vector is employed. The levels of expression of the transgene, or for an immunogen, the level of circulating antibody, can be monitored to determine the frequency of dosage administration. Yet other methods for determining the timing of frequency of administration will be readily apparent to one of skill in the art.

An optional method step involves the co-administration to the patient, either concurrently with, or before or after administration of the viral vector, of a suitable amount of a short acting immune modulator. The selected immune modulator is defined herein as an agent capable of inhibiting the formation of neutralizing antibodies directed against the recombinant vector of this disclosure or capable of inhibiting cytolytic T lymphocyte (CTL) elimination of the vector. The immune modulator may interfere with the interactions between the T helper subsets (T_{H}i or T\hat{ }) and B cells to inhibit neutralizing antibody formation. Alternatively, the immune modulator may inhibit the interaction between THI cells and CTLs to reduce the occurrence of CTL elimination of the vector. A variety of useful immune modulators and dosages for use of same are disclosed, for example, in Yang et al., J. Virol., 70 (9) (September 1996); International Patent Application Publication No. WO 96/12406, published May 2, 1996; and International Patent Application No. PCT/US96/03035, all incorporated herein by reference

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of a viral composition can occur as a single event or over a time course of treatment. For example, one or more of a nanoparticle composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a respiratory virus.

The present disclosure encompasses pharmaceutical compositions comprising recombinant adenovirus as disclosed above, so as to facilitate administration and promote stability of the recombinant adenovirus. For example, a recombinant adenovirus of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”).

Also provided are kits. Such kits can include an agent or recombinant adenovirus described herein and, in certain embodiments, instructions for administration. Such kits can facilitate performance of the methods described herein. When supplied as a kit, the different components of the composition can be packaged in separate containers and admixed immediately before use. Components include, but are not limited to compositions and pharmaceutical formulations comprising a nanoparticle composition or recombinant adenovirus, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

As various changes could be made in the above-described materials and methods without departing from the scope of the disclosure, it is intended that all matter contained in the above description and in the examples given below, shall be interpreted as illustrative and not in a limiting sense.

Additional objects, advantages, and novel features of this disclosure will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Construction of ChAd vector to express SARS-COV-2 Spike protein: Simian Ad36 vector (ChAd) was obtained from the Penn Vector Core of the University of Pennsylvania. The ChAd genome was engineered with deletions in the E1 and E3B region (GenBank: FJ025917.1; nucleotides 455-3026 and 30072-31869, respectively). A modified human cytomegalovirus (CMV) major immediate early promoter sequence was incorporated in place of the E1 genes in counterclockwise orientation on complementary DNA strand. CMV modification included an addition of two copies of the Tet operator 2 (TetO2) sequence (T-REx system) inserted in tandem (5′-TCT CTA TCA CTG ATA GGG AGA TCT CTA TCA CTG ATA GG GA-3′ SEQ ID NO: 4) between the TATA box and the mRNA start (GenBank: MN920393, nucleotides 174211-174212). SARS-COV-2 Spike(S) glycoprotein gene (encoding a prefusion stabilized mutant with two proline substitutions at residues K986 and V987 that stabilize the prefusion form of S was cloned into a unique Pmel site under the CMV-tetO2 promoter control in pSAd36 genomic plasmid to generate pSAd36-S. In parallel, a pSAd36-control carrying an empty CMV-tetO2 cassette with no transgene also was generated. The pSAd36-S and pSAd-control plasmids were linearized with PacI restriction enzyme to liberate viral genomes for transfection into T-REx™-293 cells (Invitrogen). The rescued replication incompetent ChAd-SARS-COV-2-S and ChAd-Control vectors were scaled up in 293 cells and purified by CsCl density-gradient ultracentrifugation. Viral particle concentration in each vector preparation was determined by spectrophotometry at 260 nm as described.

Construction of ChAd vector to express SARS-COV-2 Spike gene: FIG. 2 schematic illustrates one particular construction of ChAd vector for expressing SARS-CoV-2 spike protein. A similar construct can be used to include one or more SARS-COV-2 (S)-protein genes in rAd vectors of the present disclosure.

Adenoviral Vectors for intranasal/inhalation or intramuscular vaccine: PCT Patent Application Publication No. WO 2021/247567, which is incorporated herein in its entirety, describes a single-dose, intranasally (IN)-delivered chimpanzee Adenovirus (simian Ad-36)-based SARS-COV-2 vaccine (ChAd-SARS-COV-2-S) encoding a pre-fusion stabilized S protein that induced robust humoral, cell-mediated, and mucosal immune responses and limited upper- and lower-airway infection in K18-hACE2 transgenic mice, hamsters, and non-human primates. See, also, Hassan et al., 2021, Cell Reports, 36, 109452. This vaccine, which has advanced to human clinical trials (BBV154, Clinical Trial NCT04751682), differs from ChAdOx1 nCOV-19, a chimpanzee Ad-23-based SARS-COV-2 vaccine, which was previously granted emergency use in some countries. The potential utility of ChAd-SARS-COV-2-S, its dose response, durability, and cross-protective activity in mice including effects on upper- and lower-airway infection has been addressed. At approximately 9 months after IN immunization, neutralizing antibody and anti-S protein IgG and IgA levels in serum of ChAd-SARS-COV-2-S-vaccinated animals remained high and inhibited infection with SARS-COV-2 strains displaying B.1.351, and B.1.1.28 spike proteins. Highly COVID-19 susceptible K18-hACE2 transgenic mice were fully protected against upper and lower respiratory tract infection after challenge with a SARS-COV-2 virus displaying B.1.351 spike proteins.

Some aspects of the disclosure utilize adenoviral vectors to deliver quadrivalent flu antigens (HA from the latest recommended vaccine strains) and bivalent SARS-CoV-2 (Spike protein antigen from recommended SARS-COV-2 strains) as a combined flu-COVID vaccine.

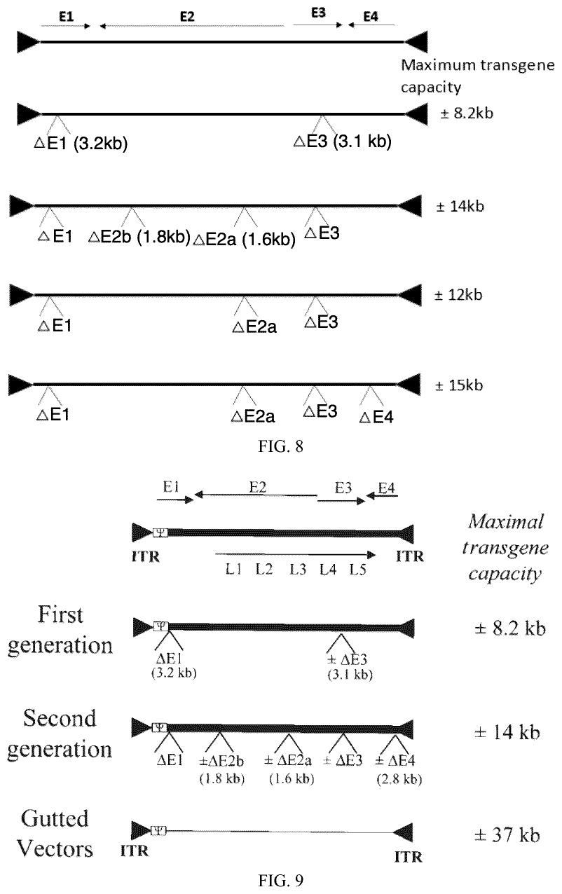

Schematic of the WT and 1-4^{th} generation Ad5 which are used in producing rAD vectors of the disclosure are shown in FIGS. 3-7, which are referred to herein as first, second, third, and fourth generation Ad5, respectively. Maximum transgene carrying capacity of 1^{st}-4^{th} generation human Ad5 vector are shown in FIG. 8. Gutted vectors can also be used to substantially improve the antigen carrying capacity of the adenoviral vectors. FIG. 9. See, also, Danthinne et al., Gene Therapy, 2000, 7, pp. 1707-1714.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A recombinant adenovirus adapted for use in preventing infection or transmission, or reducing severity of disease caused by influenza and/or SARS-Cov-2 virus in a subject, said recombinant adenovirus comprising an oligonucleotide that encodes an immune response stimulating antigen in the subject, wherein said recombinant adenovirus comprises at least two different oligonucleotides, wherein each of said oligonucleotide is selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is a different strain compared to said first influenza B virus;
(v) an oligonucleotide that encodes a spike(S) protein or an immunogenic portion, variant, mutant, or fragment thereof of a first SARS-COV-2 virus; and
(vi) an oligonucleotide that encodes an S protein or an immunogenic portion, variant, mutant, or fragment thereof of a second SARS-COV-2 virus, wherein said second SARS-COV-2 virus is a different strain compared to said first SARS-COV-2 virus;
and wherein an adenovirus of said recombinant adenovirus is selected from the group consisting of:
(i) a first, second, or third-generation human adenovirus 5;
(ii) a rare-serotype adenovirus;
(iii) a nonhuman adenovirus; and
(iv) a combination thereof.

2. The recombinant adenovirus of claim 1, wherein said recombinant adenovirus comprises:
(a) at least one of said oligonucleotide of (i)-(iv); and
(b) at least one said oligonucleotide of (v) and (vi).

3. The recombinant adenovirus of claim 1, wherein at least one of said oligonucleotides encodes at least one of 18 hemagglutinin (HA) subtypes 1-18 or an immunogenic portion, variant, mutant, or fragment thereof.

4. The recombinant adenovirus of claim 1, wherein at least one of said oligonucleotides encodes at least one of 10 neuraminidase (NA) subtypes 1-10 or an immunogenic portion, variant, mutant, or fragment thereof.

5. The recombinant adenovirus of claim 1, wherein at least one of said oligonucleotides encodes B/Yamagata/16/88 HA or an immunogenic portion, variant, mutant, or fragment thereof.

6. The recombinant adenovirus of claim 1, wherein at least one of said oligonucleotides encodes B/Victoria/2/87 HA or an immunogenic portion, variant, mutant, or fragment thereof.

7. The recombinant adenovirus of claim 1, wherein at least one of said oligonucleotides encodes said S-protein of said first SARS-COV-2 virus selected from the group consisting of SARS-COV-2 variants B.1.1.7, B.1.351, B1.1.28-P.1, B.1.617.2, B.1.1.529, BA.4, BA.5, BQ1.1, XBB.1.5, BA.2.75.2, and an immunogenic portion, variant, mutant, and fragment thereof.

8. The recombinant adenovirus of claim 1, wherein at least one of said oligonucleotides encodes said S-protein of said second SARS-COV-2 virus.

9. The recombinant adenovirus of claim 1, wherein said rare-serotype adenovirus comprises Ad11, Ad26, Ad35, Ad48, Ad49, Ad50, or a combination thereof.

10. The recombinant adenovirus of claim 1, wherein said nonhuman adenovirus comprises simian Ad36, bovine Ad3, canine Ad2, porcine Ad3, or a combination thereof.

11. A plasmid comprising a genome of an adenovirus, wherein said genome of the adenovirus has been modified such that said genome comprises a transgene that is operatively linked to expression control sequences which direct transcription, translation, and/or expression in a host cell, and wherein said transgene comprises at least two different oligonucleotides, wherein each of said oligonucleotide is independently selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is a different strain than said first influenza B virus;
(v) an oligonucleotide that encodes a spike protein (S-protein) or an immunogenic portion, variant, mutant, or fragment thereof of a first SARS-COV-2 virus; and
(vi) an oligonucleotide that encodes an S-protein or an immunogenic portion, variant, mutant, or fragment thereof of a second SARS-COV-2 virus, wherein said second SARS-COV-2 virus is a different strain than said first SARS-COV-2 virus.

12. The plasmid of claim 11, wherein said genome is derived from an adenovirus selected from the group consisting of:
(i) a first, second, third, or fourth-generation human adenovirus 5;
(ii) a rare-serotype adenovirus;
(iii) a nonhuman adenovirus; and
(iv) a combination thereof.

13. The plasmid of claim 12, wherein said adenovirus is a nonhuman adenovirus.

14. The plasmid of claim 13, wherein said nonhuman adenovirus comprises simian Ad36, bovine Ad3, canine Ad2, porcine Ad3, or a combination thereof.

15. The plasmid of claim 12, wherein said nonhuman adenovirus comprises simian Ad36.

16. The plasmid of claim 15, wherein said genome of simian Ad36 lacks a native E1 and optionally, the E3 or E3B locus.

17. A method for reducing or preventing an incidence of or reducing a severity of influenza virus infection, SARS-COV-2 virus infection, or a combination thereof in a subject, said method comprising administering a therapeutically effective amount of a polycistronic vaccine to the subject, wherein said polycistronic vaccine comprises a recombinant adenovirus (rAd) vector or a pharmaceutical composition comprising said rAd vector, wherein said rAd vector comprises a genome of an adenovirus that has been modified such that said genome comprises (i) a first extraneous oligonucleotide encoding at least one immune response stimulating antigen of an influenza virus and (ii) a second extraneous oligonucleotide encoding at least one immune response stimulating antigen of a SARS-COV-2 virus.

18. The method of claim 17, wherein said first extraneous oligonucleotide is selected from the group consisting of:
(i) an oligonucleotide that encodes a hemagglutinin of influenza A virus or an immunogenic portion, variant, mutant, or fragment thereof;
(ii) an oligonucleotide that encodes a neuraminidase of influenza A virus or an immunogenic portion, variant, mutant, or fragment thereof;
(iii) an oligonucleotide that encodes a hemagglutinin of a first influenza B virus or an immunogenic portion, variant, mutant, or fragment thereof;
(iv) an oligonucleotide that encodes a hemagglutinin of a second influenza B virus or an immunogenic portion, variant, mutant, or fragment thereof, wherein said second influenza B virus is of a different strain than said first influenza B virus; and
(v) a combination of two of more said oligonucleotides of (i)-(iv).

19. The method of claim 17, wherein said second extraneous oligonucleotide is selected from the group consisting of:
(vi) an oligonucleotide that encodes a spike protein (S-protein) or an immunogenic portion, variant, mutant, or fragment thereof of a first SARS-COV-2 virus;
(vii) an oligonucleotide that encodes an S-protein or an immunogenic portion, variant, mutant, or fragment thereof of a second SARS-COV-2 virus, wherein said second SARS-COV-2 virus is of a different strain than said first SARS-COV-2 virus; and
(viii) a combination of said oligonucleotides of (vi) and (vii).

20. The method of claim 17, wherein said polycistronic vaccine is administered to said subject via intramuscular, intranasal, or inhalation route.
